SEQUENCE LISTING (15 sequences)

SEQ ID NO: 1 - DNA, 1199 bp, Artificial Sequence
  Yarrowia lipolytica codon optimized sequence
gaatgcagcggcccagccggccatggcccaggtgcagctgcaggtcgacctcgagtggcggcggaggctctggcggaggcggatctggcggcggtggcagtgcacaggtccaactgcaggagctcgatatcaaacgggcggccgcagagcagaagctgatctctgaggaagatctgtccggcggaggcggctccggtggcggcggttctggcggtggcggctctcatatgtctgccaagtcctctttcatctctaccaccaccaccgacctgacctctatcaacacctctgcctactctaccggctctatctctaccgtggagaccggcaaccgaaccacctctgaagtgatctctcacgtggtgaccacttctaccaagctgtctcccaccgccaccacctccctgaccattgcccagacctctatctactccaccgactccaacatcaccgtgggcaccgacatccacaccacctccgaggtcatttccgacgtggagaccatctcccgagagaccgcctctaccgtggtggccgctcctacctctaccaccggctggaccggcgccatgaacacctacatctctcagttcacctcttcttccttcgccaccatcaactctacccccatcatctcttcctctgccgtgttcgagacctctgacgcctctatcgtgaacgtccacaccgagaacattaccaacaccgccgctgttccctctgaggaacccacctttgtgaacgccacccgaaactccctgaactctttctgttcttctaagcagccctcctctccctcttcctacacctcttcccccctggtgtcctctctgtctgtgtctaagaccctgctgtctacctctttcaccccctctgtgcccacctctaacacctacattaagaccaagaacaccggctacttcgagcacaccgccctgaccacctcttctgtgggcctgaactccttctctgagaccgccgtgtcctctcagggcaccaagatcgacacctttctggtctcctccctgatcgcctacccctcttctgcctctggctctcagctgtctggcatccagcagaacttcacctctacctccctgatgatctctacctacgagggcaaggcctctatcttcttctctgccgagctgggctctatcatcttcctgctgctgtcttacctgctgttctaacctagg

SEQ ID NO: 2 - DNA, 446 bp, Artificial Sequence
  Yarrowia lipolytica codon optimized sequence
gaatgcagcggcccagccggccatggcccaggtgcagctgcaggtcgacctcgagtggcggcggaggctctggcggaggcggatctggcggcggtggcagtgcacaggtccaactgcaggagctcgatatcaaacgggcggccgcagagcagaagctgatctctgaggaagatctgtccggcggaggcggctccggtggcggcggttctggcggtggcggctctcatatgcaggaactgaccaccatctgcgagcagattccctctcccaccctggagtctaccccctactctctgtctaccaccaccatcctggccaacggcaaggccatgcagggcgtgttcgagtactacaagtctgtgaccttcgtgtctaactgtggctctcacccctctaccacctctaagggctctcccatcaacacccagtacgtgttctaacctagg

SEQ ID NO: 3 - DNA, 569 bp, Artificial Sequence
  Yarrowia lipolytica codon optimized sequence
gaatgcagcggcccagccggccatggcccaggtgcagctgcaggtcgacctcgagtggcggcggaggctctggcggaggcggatctggcggcggtggcagtgcacaggtccaactgcaggagctcgatatcaaacgggcggccgcagagcagaagctgatctctgaggaagatctgtccggcggaggcggctccggtggcggcggttctggcggtggcggctctcatatgggcaacggttacgccgtcgacgacaactccaagtgcgaggacgacggaatccccttcggcgcctacgctgttgctgacacctccgcagagtcttctgccgcccccgcctcttctgccgccgctgccgagtcctctgccgccccctcttccgctgctgaggccaagcccaccgctggaggtaacaccggcgccgtcgtcacccagatcggtgacggccagatccaggctcccccctctgctcctcccgctgcccccgagcaggccaacggcgccgtctctgtcggtgtttctgccgccgctctcggtgtcgctgccgccgctctcctcatttaacctagg

SEQ ID NO: 4 - DNA, 470 bp, Artificial Sequence
  Yarrowia lipolytica codon optimized sequence
gaatgcacaggaactgaccaccatctgcgagcagattccctctcccaccctggagtctaccccctactctctgtctaccaccaccatcctggccaacggcaaggccatgcagggcgtgttcgagtactacaagtctgtgaccttcgtgtctaactgtggctctcacccctctaccacctctaagggctctcccatcaacacccagtacgtgttctcttctggcggcggaggctctggcggaggcggatctggtggcggaggatctgcggcccagccggccatggcccaggtgcagctgcaggtcgacctcgagtggaggcggcggatctggcggtggcggctccggcggtggaggcagtgcacaggtccaactgcaggagctcgatatcaaacgggcggccgcagagcagaagctgatctctgaggaagatctgcgaaccggccaccaccaccaccaccactaacctagg

SEQ ID NO: 5 - DNA, 747 bp, Artificial Sequence
  Yarrowia lipolytica codon optimized sequence
ggcccagccggccgaggtgcagctggtcgagtctggcggcggactggtgcagcccggtggctctctgcgactgtcttgtgccgcctctggcttcaacatcaaggacacctacatccactgggtgcgacaggctcccggaaagggcctggagtgggtggcccgaatctaccccaccaacggctacacccgatacgccgactctgtgaagggccgattcaccatctctgccgacacctctaagaacaccgcctacctgcagatgaactctctgcgagccgaggacaccgctgtgtactactgttctcgatggggaggcgacggcttctacgccatggactactggggccagggcaccctggtgaccgtgtcctctggcggaggcggctccggcggaggcggatctggtggcggaggctctgacatccagatgacccagtctccctcttctctgtctgcctctgtgggcgaccgagtgaccatcacctgtcgagcctctcaggacgtgaacaccgccgtggcctggtatcagcagaagcccggcaaggcccccaagctgctgatctactctgcctctttcctgtactctggcgtgccctctcgattctctggctctcgatctggcaccgacttcaccctgaccatctcttctctgcagcctgaggatttcgccacctactactgtcagcagcactacaccaccccccccaccttcggccagggaaccaaggtggagatcaaggcggccgc

SEQ ID NO: 6 - DNA, 756 bp, Artificial Sequence
  Yarrowia lipolytica codon optimized sequence
ggcccagccggccgacgtgaagctggacgagactggaggaggcctggtgcagcccggacgacccatgaagctgtcttgtgtggcctctggcttcaccttctctgactactggatgaactgggtgcgacagtctcccgagaagggcctggagtgggtggcccagatccgaaacaagccctacaactacgagacctactactctgactctgtgaagggccgattcaccatgtcccgagatgactctaagtcctctgtgtacctgcagatgaacaacctgcgagtggaggacatgggcatctactactgtaccggctcttactacggcatggactactggggccagggcacctctgtgaccgtgtcctctggcggcggaggctctggcggaggcggatctggtggcggaggatctgacgtggtgatgacccagacccccctgtctctgcccgtgtctctgggcgaccaggcctctatctcttgtcgatcttctcagtctctggtccactctaacggcaacacctacctgcgatggtatctgcagaagcccggccagtctcccaaggtgctgatctacaaggtgtctaaccgattctctggcgtgcccgaccgattctccggctctggctctggcaccgacttcaccctgaagatctcccgagtggaggccgaggacctgggcgtgtacttctgttctcagtctacccacgtgccctggaccttcggcggaggcaccaagctggagatcaaggcggccgc

SEQ ID NO: 7 - DNA, 738 bp, Artificial Sequence
  Yarrowia lipolytica codon optimized sequence
ggcccagccggcccaggtgcagctgcaggaatctggccccggactggtggccccctctcagtctctgtctatcacctgtaccgtgtctggcttctctctgaccggctacggcgtgaactgggtgcgacagccccctggcaagggcctggagtggctgggcatgatctggggcgacggcaacaccgactacaactctgccctgaagtctcgactgtctatctctaaggacaactctaagtctcaggtgttcctcaagatgaactctctccacaccgacgacaccgcccgatactactgtgcccgagagcgagactaccgactggactactggggccagggcaccaccgtgaccgtgtcctctggcggtggaggctctggcggaggcggatctggtggcggaggatctgacatcgagctgacccagtctcccgcctctctgtctgcctctgtgggcgagaccgtgaccatcacctgtcgagcctctggcaacatccacaactacctggcctggtatcagcagaagcagggcaagtctccccagctgctggtgtactacaccaccaccctggccgacggcgtgccctctcgattctctggctctggatctggcacccagtactccctgaagatcaactccctgcagcccgaggacttcggctcttactactgtcagcacttctggtctaccccccgaaccttcggcggaggcaccaagctggagatcaagcgagcggccgc

SEQ ID NO: 8 - DNA, 738 bp, Artificial Sequence
  Yarrowia lipolytica codon optimized sequence
ggcccagccggcccaggtgcagctgcaggaatctggccccggactggtggccccctctcagtctctgtctatcacctgtaccgtgtctggcttctctctgaccggctacggcgtgaactgggtgcgacagctgcctggcaagggcctggagtggctgggcatgatctggggcgacggcaacaccgcctacaactctgccctgaagtctcgactgtctatctctaaggacaactctaagtctcaggtgttcctcaagatggactctctccacaccgacgacaccgcccgatactactgtgcccgagagcgagactaccgactggactactggggccagggcaccaccgtgaccgtgtcctctggcggtggaggctctggcggaggcggatctggtggcggaggatctgacatcaagctgacccagtctcccgcctctctgtctgcctctgtgggcgagaccgtgaccatcacctgtcgagcctctggcaacacccacaactacctggcctggtatcagcagaagcagggcaagtctccccagctgctggtgtactacaccaccaccctggccgacggcgtgccctctcgattctctggctctggatctggcacccagtactccctgaagatcaactccctgcagcccgaggacttcggctcttactactgtcagcacttctggtctaccccccgatctttcggcggaggcaccaagctggagatcaagcgagcggccgc

SEQ ID NO: 9 - DNA, 675 bp, Artificial Sequence
  Yarrowia lipolytica codon optimized sequence
ggcccagccggccgacgtgaagctggacgagactggaggaggcctggtgcagcccggacgacccatgaagctgtcttgtgtggcctctggcttcaccttctctgactactggatgaactgggtgcgacagtctcccgagaagggcctggagtgggtggcccagatccgaaacaagccctacaactacgagacctactactctgactctgtgaagggccgattcaccatgtcccgagatgactctaagtcctctgtgtacctgcagatgaacaacctgcgagtggaggacatgggcatctactactgtaccggctcttactacggcatggactactggggccagggcacctctgtgaccgtgtcctctgctagcaccaagggaccttctgtgtttcctctggccccctcttctaagtctacctctggtggaactgctgctctgggatgtctggtgaaggactactttcctgagcctgtgactgtgtcttggaactctggcgctctgacttctggtgttcacaccttccctgctgttctgcagtcctctggactgtactctctctcttctgtggtgaccgtgccttcttcttctctgggaacccagacctacatctgtaacgtgaaccacaagccctctaacactaaggtggacaagcgagtggagcctgcggccgc

SEQ ID NO: 10 - DNA, 678 bp, Artificial Sequence
  Yarrowia lipolytica codon optimized sequence
ggcccagccggccgacgtggtgatgacccagacccccctgtctctgcccgtgtctctgggcgaccaggcctctatctcttgtcgatcttctcagtctctggtccactctaacggcaacacctacctgcgatggtatctgcagaagcccggccagtctcccaaggtgctgatctacaaggtgtctaaccgattctctggcgtgcccgaccgattctccggctctggctctggcaccgacttcaccctgaagatctcccgagtggaggccgaggacctgggcgtgtacttctgttctcagtctacccacgtgccctggaccttcggcggaggcaccaagctggagatcaagcgtacggtggctgctccttctgtgttcattttccccccctctgacgagcagctgaagtctggaactgcttctgttgtgtgcctgctgaacaacttttacccccgagaggctaaggttcagtggaaggtggacaacgctctgcagtctggaaactctcaggagtctgttactgagcaggactctaaggactcgacctactctctctcttctaccctgaccctgtctaaggctgactacgagaagcataaggtgtacgcttgtgaggttacccatcagggactgtcctctcccgtgaccaagtcttttaaccgaggcgagtgcgcggccgc

SEQ ID NO: 11 - DNA, 681 bp, Artificial Sequence
  Yarrowia lipolytica codon optimized sequence
ggcccagccggccgaggtgcagctggtcgagtctggcggcggactggtgcagcccggtggctctctgcgactgtcttgtgccgcctctggcttcaacatcaaggacacctacatccactgggtgcgacaggctcccggaaagggcctggagtgggtggcccgaatctaccccaccaacggctacacccgatacgccgactctgtgaagggccgattcaccatctctgccgacacctctaagaacaccgcctacctgcagatgaactctctgcgagccgaggacaccgctgtgtactactgttctcgatggggaggcgacggcttctacgccatggactactggggccagggcaccctggtgaccgtgtcctctgctagcaccaagggaccttctgtgtttcctctggccccctcttctaagtctacctctggtggaactgctgctctgggatgtctggtgaaggactactttcctgagcctgtgactgtgtcttggaactctggcgctctgacttctggtgttcacaccttccctgctgttctgcagtcctctggactgtactctctctcttctgtggtgaccgtgccttcttcttctctgggaacccagacctacatctgtaacgtgaaccacaagccctctaacactaaggtggacaagcgagtggagcctgcggccgc

SEQ ID NO: 12 - DNA, 655 bp, Artificial Sequence
  Yarrowia lipolytica codon optimized sequence
ggcccagccggccgacatccagatgacccagtctccctcttctctgtctgcctctgtgggcgaccgagtgaccatcacctgtcgagcctctcaggacgtgaacaccgccgtggcctggtatcagcagaagcccggcaaggcccccaagctgctgatctactctgcctctttcctgtactctggcgtgccctctcgattctctggctctcgatctggcaccgacttcaccctgaccatctcttctctgcagcctgaggatttcgccacctactactgtcagcagcactacaccaccccccccaccttcggccagggaaccaaggtggagatcaagcgtacggtggctgctccttctgtgttcattttccccccctctgacgagcagctgaagtctggaactgcttctgttgtgtgcctgctgaacaacttttacccccgagaggctaaggttcagtggaaggtggacaacgctctgcagtctggaaactctcaggagtctgttactgagcaggactctaaggactcgacctactctctctcttctaccctgaccctgtctaaggctgactacgagaagcataaggtgtacgcttgtgaggttacccatcagggactgtcctctcccgtgaccaagtcttttaaccgaggcgagtgc

SEQ ID NO: 13 - protein, 13 aa, Artificial Sequence
  Epitope tag
GAPVPYPDPLEPR

SEQ ID NO: 14 - protein, 15 aa, Artificial Sequence
  Peptide linker
GGGGSGGGGSGGGGS

SEQ ID NO: 15 - protein, 10 aa, Artificial Sequence
  Peptide linker
GSGSGSGSGS

(19) United States
(12) Patent Application Publication
(10) Pub. No.: US 2013/0096281 A1
(43) Pub. Date: Apr. 18, 2013

(54) METHODS AND COMPOSITIONS FOR DISPLAYING A POLYPEPTIDE ON A YEAST CELL SURFACE

(71) Applicants: Stefan Ryckaert, Sint-Amandsberg (BE); Guillaume Lerondel, Honfleur (14) (FR)

(72) Inventors: Stefan Ryckaert, Sint-Amandsberg (BE); Guillaume Lerondel, Honfleur (14) (FR)

(73) Assignee: OXYRANE UK LIMITED, Manchester (GB)

(21) Appl. No.: 13/574,126

(22) PCT Filed: Jan. 21, 2011
(86) PCT No.: PCT/IB2011/000227

§ 371 Date: Nov. 12, 2012

Related U.S. Application Data
(60) Provisional application No. 61/297,093, filed on Jan. 21, 2010.

(51) Int. Cl. C12N 15/81

(52) U.S. Cl. CPC C12N 15/815
USPC 530/387.3; 435/320.1; 435/69.6; 435/7.31

(57) ABSTRACT

Provided herein are methods and compositions for use in displaying a polypeptide (e.g., an antibody polypeptide or an antibody polypeptide fragment) on the surface of a yeast cell. Exemplary yeast that can be used in conjunction with various methods and compositions disclosed herein include those of the genus Yarrowia, e.g., Yarrowia lipolytica.

### TECHNICAL FIELD

Provided herein are methods and compositions for use in displaying a polypeptide (e.g., an antibody polypeptide or an antibody polypeptide fragment) on the surface of a yeast cell. Exemplary yeast that can be used in conjunction with various methods and compositions disclosed herein include those of the genus Yarrowia, e.g., Yarrowia lipolytica.

### BACKGROUND

High affinity reagents, e.g., antibodies or fragments thereof, are useful tools both for clinical and research applications. A number of in vitro and in vivo platforms have been used for the isolation and characterization of antibodies, including ribosome display, phage display, and periplasmic expression in E. coli. Another platform that has been used is yeast cell surface display (YSD).

Compositions and methods for displaying antibodies and fragments thereof on the cell surface of a Yarrowia strain would be advantageous.

### SUMMARY

Provided herein are methods and compositions for use in displaying a polypeptide (e.g., an antibody polypeptide or an antibody polypeptide fragment) on the surface of a yeast cell. Exemplary yeast that can be used in conjunction with various methods and compositions disclosed herein include those of the genus Yarrowia, e.g., Yarrowia lipolytica.

In certain embodiments, compositions provided herein comprise an expression cassette comprising a promoter operably linked to a fusion sequence, which fusion sequence comprises a first nucleic acid sequence comprising a nucleotide sequence encoding an anchor polypeptide fused in frame to a second nucleic acid sequence comprising a nucleotide sequence encoding an antibody polypeptide or antibody polypeptide fragment. In certain embodiments, compositions provided herein comprise an expression cassette comprising a promoter operably linked to a first nucleic acid sequence, which first nucleic acid sequence comprises an anchor nucleotide sequence encoding an anchor polypeptide, wherein the first nucleic acid sequence can be expressed as a first fusion partner in a fusion polypeptide comprising a second fusion partner of interest encoded by a second nucleic acid sequence. In certain embodiments, an expression cassette further comprising a second nucleic sequence encoding the second fusion partner of interest, e.g., all or part of a restriction site. In certain embodiments, the second fusion partner of interest comprises an antibody polypeptide or antibody polypeptide fragment. In certain embodiments, an antibody polypeptide fragment is a scFv fragment, a heavy chain of a Fab fragment, or a light chain of a Fab fragment.

In certain embodiments, the first nucleic acid sequence of an expression cassette is fused 3′ to the second nucleic acid sequence, such that a fusion polypeptide produced from the fusion sequence comprises an N-terminal antibody polypeptide or antibody polypeptide fragment and a C-terminal anchor polypeptide. In certain embodiments, the first nucleic acid sequence of an expression cassette is fused 5′ to the second nucleic acid sequence, such that a fusion polypeptide produced from the fusion sequence comprises an N-terminal anchor polypeptide and a C-terminal antibody polypeptide or antibody polypeptide fragment.

In certain embodiments, an expression cassette comprises a constitutive promoter. In certain embodiments, an expression cassette comprises an inducible promoter, e.g., a POX2 or LIP2 promoter. In certain embodiments, an expression cassette comprises a semi-inducible promoter, e.g. an ph4d promoter.

In certain embodiments, an expression cassette comprises a leader nucleic acid sequence comprising a nucleotide sequence encoding a leader polypeptide, wherein the leader nucleic acid sequence is fused in frame, 5′ to the first and second nucleic acid sequences. Exemplary leader nucleic acid sequences include, without limitation, LIP2 pre, LIP2 prepro, XPR2 pre, and XPR2 prepro.

In certain embodiments, an expression cassette comprises a linker nucleic acid sequence comprising a nucleotide sequence encoding a linker polypeptide. For example, the linker nucleic acid sequence can be fused in frame between the first and second nucleic acid sequences. In certain embodiments, the antibody polypeptide comprises an scFv antibody polypeptide, and the linker nucleic acid sequence is fused in frame between a heavy chain nucleic acid sequence encoding variable region and a light chain nucleic acid sequence encoding a variable region of the scFv polypeptide. Non-limiting examples of linker polypeptides include (Gly4Ser)₃ or (GlySer)₅.

In certain embodiments, an expression cassette comprises one or more nucleic acid sequences comprising a nucleotide sequence encoding one or more epitope tags. Exemplary epitope tags include, without limitation, c-Myc, V5, hexahistidine, glutathione-5-transferase, streptavidin, biotin, hemagglutinin, Flag-tag, and E-tag.

In certain embodiments, an expression cassette comprises an anchor polypeptide. Non-limiting examples of anchor polypeptides include an Aga1p polypeptide or fragment thereof, an Aga2p polypeptide or fragment thereof, and a Sag1p polypeptide or fragment thereof.

In certain embodiments, an expression cassette comprises an antibody polypeptide or antibody polypeptide fragment, an anchor polypeptide, or both that are codon optimized for expression in a Yarrowia cell.

In certain embodiments, compositions provided herein comprise a vector that comprises any of the expression cassettes described above. In certain embodiments, a vector comprises a zeta element. Exemplary zeta elements include, without limitation, long terminal repeats of a retrotransposon such as, e.g., a Ylt1 or Tyl6 retrotransposon. In certain embodiments, a vector comprises one or more autosomal replication elements, e.g., autosomal replication elements comprising a centromere (CEN) and an origin of replication (ORI). Exemplary centromeres include, without limitation, CEN1 and CEN3. Exemplary origins of replication include, without limitation, ORI1068 or ORI3018. In certain embodiments, a vector comprises an autonomously replicating sequence (ARS), which comprises a centromere and an origin of replication. Exemplary ARSs include, without limitation, ARS18 and ARS68. In certain embodiments, a vector comprises one or more nucleic acid sequences comprising a nucleotide sequence encoding one or more selectable markers. Non-limiting examples of selectable markers include LEU2, URA3d1, ADE2, Lys, Arg, Gut, Tip, G3p, and hph.

In certain embodiments, methods provided herein comprise methods for displaying an antibody polypeptide or antibody polypeptide fragment on the surface of a Yarrowia cell. For example, an antibody polypeptide or antibody polypeptide fragment may be displayed on the surface of a Yarrowia cell by introducing into a first Yarrowia cell a first vector comprising a promoter operably liked to a fusion sequence comprising a first nucleic acid sequence comprising a nucleotide sequence encoding an antibody polypeptide or antibody polypeptide fragment fused in frame to a second nucleic acid sequence comprising a nucleotide sequence encoding an anchor polypeptide, and incubating the first Yarrowia cell for a time and under Yarrowia cell operating conditions. Exemplary first vectors include, without limitation, any of the vectors described above. In certain embodiments, an antibody polypeptide fragment is a scFv fragment, a heavy chain of a Fab fragment, or a light chain of a Fab fragment. In certain embodiments, methods may comprise introducing into the first Yarrowia cell a second vector comprising a second promoter operably linked to a nucleic acid sequence encoding a light chain of a Fab fragment or a heavy chain of a Fab fragment. In certain embodiments, the first Yarrowia cell is haploid, and the step of introducing the second vector comprises mating the first haploid Yarrowia cell comprising the first vector with a second haploid Yarrowia cell comprising the second vector, the first and second Yarrowia cells being of opposite mating types.

In certain embodiments, the first nucleic acid sequence is fused 5′ to the second nucleic acid sequence, such that a fusion polypeptide produced from the fusion sequence comprises an N-terminal antibody polypeptide or antibody polypeptide fragment thereof and a C-terminal anchor polypeptide. In certain embodiments, the first nucleic acid sequence is fused 3′ to the second nucleic acid sequence, such that a fusion polypeptide produced from the fusion sequence comprises an N-terminal anchor polypeptide and a C-terminal antibody polypeptide or antibody polypeptide fragment.

In certain embodiments, a Yarrowia cell operating condition comprises a low induction temperature, e.g., a temperature between about 15 degrees Celsius and 25 degrees Celsius. A non-limiting low induction temperature comprises a temperature of about 20 degrees Celsius. In certain embodiments, a Yarrowia cell operating condition comprises a short induction time, e.g., about 24 hours or less, about 16 hours or less, or about 16 hours. In certain embodiments, a Yarrowia cell operating condition comprises a low pH, e.g., a pH of between about 2 and about 4, or a pH of about 3. In certain embodiments, a Yarrowia cell operating condition comprises high aeration conditions, e.g., incubation in a shake flask. In certain embodiments, a Yarrowia cell operating condition comprises incubation in minimal medium, e.g., a medium that lacks yeast extract, bactopeptone, or both.

In certain embodiments, the first vector is integrated into the Yarrowia genome. In certain embodiments, the Yarrowia cell expresses a chaperone, e.g., a protein disulfide isomerase, and/or Kar2/Bip.

In certain embodiments, compositions provided herein comprise an antibody polypeptide or antibody polypeptide fragment obtained by any of the methods described above. In certain embodiments, methods for selecting a Yarrowia cell comprising an antibody polypeptide or antibody polypeptide fragment that binds a target polypeptide are provided. For example, a Yarrowia cell comprising an antibody polypeptide or antibody polypeptide fragment that binds a target polypeptide may be selected by providing a parent Yarrowia cell (e.g., a Yarrowia cell is produced by any of the methods described above) displaying on its surface an antibody polypeptide or antibody polypeptide fragment, contacting the parent Yarrowia cell with the test polypeptide, and selecting the parent Yarrowia cell if the displayed antibody polypeptide or antibody polypeptide fragment binds the target polypeptide. In certain embodiments, such methods comprise isolating the first expression cassette of the antibody polypeptide or antibody polypeptide fragment from the selected parent Yarrowia cell, introducing one or more changes in the nucleotide sequence encoding the antibody polypeptide or antibody polypeptide fragment to generate a modified expression cassette, introducing the modified expression cassette into a second Yarrowia cell that lacks the first expression cassette to generate a modified Yarrowia cell, incubating the modified Yarrowia cell for a time and under Yarrowia cell operating conditions, contacting the modified Yarrowia cell with the target polypeptide, and selecting the modified Yarrowia cell if it binds the target polypeptide with greater affinity or avidity than the parent Yarrowia cell.

In certain embodiments, kits are provided herein. In certain embodiments, kits provided herein comprise an expression cassette such as any of the expression cassettes described above. In certain embodiments, kits provided herein comprise a vector such as any of the vectors described above. In certain embodiments, kits provided herein comprise a Yarrowia cell. In certain embodiments, kits provided herein comprise written instructions for use of an expression cassette, a vector, or both.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic representation of expression plasmids and expression cassettes used for Yarrowia lipolytica display of scFv and Fab fragments. FIG. 1A shows the components and map of a Yarrowia expression plasmid for random integration. The expression of the target gene is driven by the inducible pPOX2 promoter. Different transformation markers are available to allow the creation of a fully complemented strain (Leu2, Ade2, Ura3). This plasmid was used as a template to clone the different antibody fragments. FIG. 1B shows expression cassettes for soluble expression of AGA1, scFv fragment and Fab fragment light chain ck1 domain. Synthetic cassettes were cloned into the Yarrowia expression plasmids using the shown restriction sites. Light chain variable domains can be cloned separately into the resulting plasmids, creating display plasmids of the full length Fab light chain fragment (VL-Ck1) containing light chain variable regions (VL) and light chain constant regions. FIG. 1C shows scFv antibody fragments that were cloned into Yarrowia expression plasmid using the shown restriction sites. A total of four synthetic constructs were made that allow anchorage in the different fusion modes and using the different anchorage molecules. FIG. 1D shows Fab CH1 antibody fragments (Fab fragments that contain heavy chain constant region CH1 domains) that were cloned into Yarrowia expression plasmid using the shown restriction sites. A total of four synthetic constructs were made that allow anchorage in the different fusion modes and using the different anchorage molecules. Heavy chain variable domains can be cloned separately into the resulting plasmids, creating display plasmids of the full length Fab heavy chain composed of the VH and the heavy chain constant region CH1 domain (VH-CH1). FIG. 1E shows co-transformation strategies and schematic representations of the various polypeptides that are expressed from each of the scFv and Fab fragments with their appropriate anchor polypeptides as they would be expressed on the surface of Yarrowia lipolytica cells.

FIG. 2 is a series of one-dimensional fluorescence flow cytometry (FFC) histograms depicting c-Myc-tagged scFv expression in Yarrowia lipolytica cells induced for 20 hours at 20° C. in minimal supplemented medium (MM) and rich medium (RM) both for FALCON and shake flask (SF) cultures (86%). Cells were also grown in MM at 28° C. in shake flasks. The top panels show FFC histograms for c-Myc-tagged scFv fragments, while the bottom panels show FFC histograms for strain 1T2 that expresses a full size monoclonal Herceptin antibody. Fluorescence was detected as described in Example 1 below. Shaded histograms show autofluorescence (negative control), while solid lines represent c-myc expression.

FIG. 3 is a series of one-dimensional FFC histograms depicting c-Myc-tagged scFv expression in Yarrowia lipolytica cells induced for varying amounts of time. The histograms depict the effect of induction time on surface display levels of c-Myc-tagged scFv in Yarrowia lipolytica cells. Cells were grown for 16, 20, 24, 32 and 43 hours. The relative proportion of cells expressing c-Myc decreased with longer induction times (54% after 24 hours, 19% after 32 hours, and 7% after 43 hours). Fluorescence was detected as described in Example 1. Shaded histograms show autofluorescence (negative control), while solid lines represent c-myc expression.

FIG. 4 is a series of one-dimensional FFC histograms depicting the effect of pH on surface display levels of c-Myc-tagged scFv in Yarrowia lipolytica cells. Cells were grown at pH 6.8, pH 5, and pH3 for 24 hours (top panels) and 32 hours (bottom panels). Panels on the left show background fluorescence of cells that are not expressing scFv on their surface. Panels on the right show fluorescence of cells that are expressing scFv on their surface. Fluorescence was detected as described in Example 1.

FIG. 5 is a series of one-dimensional FFC histograms depicting surface expression of two different c-Myc-tagged scFv fragments: 4-4-20 scFv (graphs below “4-4-20 scFv” label) and herceptin scFv (graphs below “herceptin scFv” label). A total of four display plasmids was created allowing display of a scFv fragment as N-terminal fusion to the C-terminal part of S. cerevisiae Sag1p (320 C-terminal AA; histograms in row labeled “A1”), N-terminal fusion to S. cerevisiae Aga2p (histograms in row labeled “A2”), N-terminal fusion to the C-terminal part of Yarrowia lipolytica CwpIp (110 C-terminal AA; histograms in row labeled “A3”) and C-terminal fusion to Aga2p (histograms in row labeled “A4”). The scFv fragments were able to bind antigen (panels in the columns labeled “ligand binding”). For ligand-binding detection, biotinylated antigen was detected with streptavidin-phycoerythrin. Fluorescence was detected as described in Example 1. For each graph, the shaded histogram represents the autofluorescence (negative control). The solid lines represent c-myc expression or ligand binding as indicated above each column.

FIG. 6 is a series of immunofluorescence micrographs of cells expressing either c-Myc-tagged 4-4-20 scFv fusion proteins (FIG. 6A) or c-Myc-tagged 4-4-20 heavy and light chain fusion proteins (FIG. 6B). Expression was detected by staining with anti-c-Myc antibody.

FIG. 7 is a series of one-dimensional FFC histograms depicting surface expression of two different c-Myc-tagged Fab fragments: 4-4-20 Fab (histograms below “4-4-20 Fab” heading) and herceptin Fab (histograms below “herceptin Fab” heading). A total of four display plasmids was created allowing display of a Fab heavy chain fragment as N-terminal fusion to the C-terminal part of S. cerevisiae Sag1p (320 C-terminal AA; histograms in row labeled “A1”), N-terminal fusion to S. cerevisiae Aga2p (histograms in row labeled “A2”), N-terminal fusion to the C-terminal part of Yarrowia lipolytica CwpIp (110 C-terminal AA; histograms in row labeled “A3”) and C-terminal fusion to Aga2p (histograms in row labeled “A4”). The Fab light chain was expressed as a soluble fragment. Heavy chain (HC) and light chain (LC) expression was detected. For ligand-binding detection, biotinylated antigen was detected with streptavidin-phycoerythrin. Fluorescence was detected as described in Example 1. For each graph, the shaded histogram represents the autofluorescence (negative control). The solid lines represent c-myc expression (indicating anchored heavy chain fragment expression), V5 expression (indicating light chain expression) or ligand binding, as indicated above each column.

FIG. 8 is a series of one-dimensional FFC histograms depicting surface expression of Herceptin Fab. The heavy chain was an N-terminal fusion to S. cerevisiae Aga2p. The light chain was solubly expressed. Heavy chain (HC) and light chain (LC) were individually detected (histograms in rows labeled “HC” and “LC”, respectively). Simultaneous labeling of HC and LC (histograms in row labeled “HC+LC”) using two color FACS analysis demonstrated the pairing of both chains on the surface of individual yeast cells. Fluorescence was detected as described in Example 1. Shaded histograms show autofluorescence (negative control), while solid lines represent either HC or LC expression, as indicated.

FIG. 9 is a pair of bar graphs depicting the effect of chaperones on Her-scFv and Her-Fab expression. WT=wild type. TEF PD=PDI (protein disulfide isomerase) expressed under control of the TEF promoter. POX2 HACI=HACI, a transcription factor that induced UPR (unfolded protein response), expressed under control of the POX2 promoter.

FIG. 10 is a series of line graphs depicting dose response curves for displayed Herceptin scFv. Three independent titrations are shown. preA1-Herceptin scFv=Herceptin scFv fused as an N-terminal fusion to the to the C-terminal 320 amino acids of S. cerevisiae Sag1p and expressed with the Lip2pre leader sequence. preproA1-Herceptin scFv=Herceptin scFv fused as an N-terminal fusion to the C-terminal 320 amino acids of S. cerevisiae Sag1p and expressed with the Lip2prepro leader sequence. preA2-Herceptin scFv=Herceptin scFv fused to as an N-terminal fusion to S. cerevisiae Aga2p and expressed with the Lip2pre leader sequence. “[Ag]”=HER2-Fc chimeric protein (antigen) concentration. The Y axis shows fraction bound, which is calculated as MFI/(MFImax-MFImin), normalized, and expressed as a percentage. Calculated kDs are shown for each titration curve.

FIG. 11 is a pair of line graphs depicting dose response curves for displayed scFv's D1.3 and mutant M3, each of which recognizes hen egg lysozyme (HEL). M3 has a 2-fold higher affinity for hen egg lysozyme than D1.3. The displayed polypeptides were expressed as Sag1p (line graph labeled “preA1 D1.3 vs M3”) and Aga2p (line graph labeled “preA2 D1.3 vs M3”) fusion polypeptides. The D1.3 or M3 displaying cells were incubated with varying concentrations of biotinylated hen egg lysozyme (X axis showing concentration in nM). Calculated kDs are shown for each titration curve.

FIG. 12 is a schematic depiction of a replicative vector used to transform Yarrowia lipolytica. The replicative vector was constructed to contain a scFv-AGA2 expression cassette driven by a pPOX2 promoter and ARS18 for replicative propagation.

FIG. 13 is a pair of histograms depicting cell surface expression of scFv-AGA2 in Yarrowia lipolytica cells transformed with a zeta-based integrative plasmid (FIG. 13A) or a replicative plasmid (FIG. 13B). The data for the replicative plasmids represents an average of ten clones. Cells transformed with the replicative vector were grown under non-selective and selective conditions. The X axis (labeled “FL2-H”) shows c-myc fluorescence signal that was recorded in channel 2 using a phycoerythrin conjugated secondary antibody. The Y axis (labeled “counts”) shows the number of cells.

FIG. 14 is a series of one-dimensional FFC histograms depicting surface expression of the single c-Myc-tagged full length trastuzumab (herceptin) IgG. A total of two display plasmids was created allowing display of a IgG heavy chain as N-terminal fusion to S. cerevisiae Aga2p (histograms in row labeled “A2”) and C-terminal fusion to Aga2p (histograms in row labeled “A4”). The IgG light chain was expressed as a soluble fragment. Heavy chain (HC) and light chain (LC) expression was detected. Fluorescence was detected as described in Example 1. FIG. 14A is a dot blot showing c-myc and V5 expression. Clearly, all cells show expression of full length heavy chain and light chain simultaneously for both N- and C-terminal fusion to AGA2. Unlabeled cells show no detection of the epitope tags. FIG. 14B shaded histograms show c-myc and V5 expression for both fusions. A drastic improvement in display efficiency can be observed (as indicated by the dotted line) for cells in which the heavy chain is fused C-terminally of the AGA2 anchor as compared to N-terminal fusion, similarly to what was observed for herceptin Fab display. FIG. 14C shows a schematic representation of the expressed HC and LC.

FIG. 15 is a line graph depicting dose response curves for two of the isolated clones (clone 13 and clone 38) from the scFv affinity maturation screening. The Kd was determined from equilibrium titration curves and compared to wild type D1.3 Kd. The Kd values were determined to be 2.2 and 1.8 nM for clone 13 and 38 respectively. This represents a 1.8 and 2.4 fold improvement, respectively, compared to wild type Kd (4.0 nM), which lies in the same range as for the M3 mutant.

### DESCRIPTION OF CERTAIN EMBODIMENTS

Provided herein are methods and compositions for use in displaying a polypeptide (e.g., an antibody polypeptide or an antibody polypeptide fragment) on the surface of a yeast cell. Exemplary yeast that can be used in conjunction with various methods and compositions disclosed herein include those of the genus Yarrowia, e.g., Yarrowia lipolytica (Y1).

### Antibody Polypeptides and Antibody Polypeptide Fragments

Any of a variety of antibody polypeptides or fragments thereof can be expressed on the surface of a yeast cell in accordance with methods and compositions described herein.

“Antibody polypeptide” as the term is used herein refers to a polypeptide that is, or is derived from, an immunoglobulin heavy chain and/or an immunoglobulin light chain polypeptide. As is known in the art, a wild-type IgG antibody generally includes two identical heavy chain polypeptides and two identical light chain polypeptides. A given antibody comprises one of five types of heavy chains, called alpha, delta, epsilon, gamma and mu, the categorization of which is based on the amino acid sequence of the heavy chain constant region. In humans, there are two subtypes of alpha constant regions and four subtypes of gamma constant regions. These different types of heavy chains give rise to five classes of antibodies, IgA (including IgA1 and IgA2 subclasses), IgD, IgE, IgG (including IgG1, IgG2, IgG3 and IgG4 subclasses) and IgM, respectively. A given antibody also comprises one of two types of light chains, called kappa or lambda, the categorization of which is based on the amino acid sequence of the light chain constant domains. In certain embodiments, methods disclosed herein provide for expression of an antibody polypeptide on the cell surface of a yeast, e.g., a Yarrowia strain such as Yarrowia lipolytica. In certain embodiments, a full length heavy chain, a full length light chain, or both are expressed in the yeast. In certain embodiments, a fragment of a full length heavy chain, a full length light chain, or both are expressed in the yeast.

“Antibody fragment” or “antibody polypeptide fragment” as the terms are used herein refer to a polypeptide derived from an antibody polypeptide molecule that does not comprise a full length antibody polypeptide as defined above, but which still comprises at least a portion of a full length antibody polypeptide. Antibody polypeptide fragments often comprise polypeptides that comprise a cleaved portion of a full length antibody polypeptide, although the term is not limited to such cleaved fragments. Since an antibody polypeptide fragment, as the term is used herein, encompasses fragments that comprise single polypeptide chains derived from antibody polypeptides (e.g. a heavy or light chain antibody polypeptides), it will be understood that an antibody polypeptide fragment may not, on its own, bind an antigen. For example, an antibody polypeptide fragment may comprise that portion of a heavy chain antibody polypeptide that would be contained in a Fab fragment; such an antibody polypeptide fragment typically will not bind an antigen unless it associates with another antibody polypeptide fragment derived from a light chain antibody polypeptide (e.g., that portion of a light chain antibody polypeptide that would be contained in a Fab fragment), such that the antigen-binding site is reconstituted. Antibody polypeptide fragments can include, for example, polypeptides that would be contained in Fab fragments, F(ab′)₂ fragments, scFv (single chain Fv) fragments, Fv fragments, diabodies, linear antibodies, multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and V_{HH} containing antibodies. It will be appreciated that “antibody fragments” or “antibody polypeptide fragments” include “antigen-binding antibody fragments” and “antigen-binding antibody polypeptide fragments.” See e.g., U.S. Pat. Nos. 7,422,890, 7,422,742, and 7,390,884, each of which is incorporated herein by reference in its entirety.

“Humanized antibody polypeptide” as the term is used herein refers to an antibody polypeptide that has been engineered to comprise one or more human variable region (light and/or heavy chain) framework regions in its variable region together with non-human (e.g., mouse, rat, or hamster) complementarity-determining regions (CDRs) of the heavy and/or light chain polypeptides and human heavy and/or light chain constant regions. In certain embodiments, a humanized antibody comprises sequences that are entirely human except for the CDR regions. Humanized antibodies are typically less immunogenic to humans, relative to non-humanized antibodies, and thus offer certain benefits in therapeutic applications. Those of ordinary skill in the art will be aware of humanized antibodies, and will also be aware of suitable techniques for generating humanized antibody polypeptides. See e.g., U.S. Pat. Nos. 7,442,772, 7,431,927, 6,872,392, and 5,585,089, each of which is incorporated herein by reference in its entirety.

“Chimeric antibody polypeptide” as the term is used herein refers to an antibody polypeptide that has been engineered to comprise at least one human constant region. The heavy and or light chain(s) can have human constant regions. Chimeric antibodies are typically less immunogenic to humans, relative to non-chimeric antibodies, and thus offer certain benefits in therapeutic applications. Those of ordinary skill in the art will be aware of chimeric antibodies, and will also be aware of suitable techniques for generating chimeric antibody polypeptides. See e.g., U.S. Pat. Nos. 7,442,772, 7,431,927, 6,872,392, and 5,585,089, each of which is incorporated herein by reference in its entirety.

In certain embodiments, an expressed antibody polypeptide or antibody polypeptide fragment is a human antibody polypeptide or fragment. In certain embodiments, an expressed antibody polypeptide or fragment thereof is a non-human antibody polypeptide or fragment thereof, e.g., a mouse or rat antibody polypeptide or fragment thereof. In certain embodiments, an expressed antibody polypeptide or fragment thereof is chimeric in that it contains human heavy and/or light chain constant regions. In certain embodiments, an expressed antibody polypeptide or fragment thereof is humanized in that it contains one or more human framework regions in the variable region together with non-human (e.g., mouse, rat, or hamster) complementarity-determining regions (CDRs) of the heavy and/or light chain.

In certain embodiments, an antibody polypeptide to be expressed on the surface of a yeast cell comprises a heavy chain polypeptide of an antibody. In certain embodiments, a fragment of a heavy chain polypeptide, e.g., that portion of the heavy chain polypeptide that would be contained in a Fab fragment (e.g., VH-CH1), an Fv fragment, or a scFv fragment, is expressed on the surface of a yeast cell. In certain embodiments, an antibody polypeptide to be expressed on the surface of a yeast cell comprises all or part of a heavy chain constant region, e.g., an Fc region, a hinge region, etc. In certain embodiments, an antibody polypeptide to be expressed on the surface of a yeast cell lacks a heavy chain constant region. In certain embodiments, an antibody polypeptide to be expressed on the surface of a yeast cell lacks a portion of the heavy chain constant region, e.g., an Fc region.

In certain embodiments, an antibody polypeptide to be expressed on the surface of a yeast cell comprises a light chain polypeptide of an antibody. In certain embodiments, a fragment of a light chain polypeptide, e.g., an Fv fragment, or a scFv fragment, is expressed on the surface of a yeast cell. In certain embodiments, an antibody polypeptide to be expressed on the surface of a yeast cell comprises a light chain constant region. In certain embodiments, an antibody polypeptide to be expressed on the surface of a yeast cell lacks a light chain constant region.

In certain embodiments, an antibody polypeptide fragment is a polypeptide that comprises an amino acid chain that is part of a Fab fragment, a F(ab′)₂ fragment, an Fv fragment, a diabody, a linear antibody, a multispecific antibody fragment such as a bispecific, a trispecific, or a multispecific antibody (e.g., a diabody, a triabody, a tetrabody), a minibody, a chelating recombinant antibody, a tribody or bibody, an intrabody, a nanobody, a small modular immunopharmaceutical (SMIP), a binding-domain immunoglobulin fusion protein, a camelid antibody, or a V_{HH} containing antibody. In certain embodiments, an antibody polypeptide fragment is a scFv fragment.

In certain embodiments, both a heavy chain antibody polypeptide or antibody polypeptide fragment and a light chain antibody polypeptide or antibody polypeptide fragment are expressed on the surface of a yeast cell. For example, a complete heavy chain antibody polypeptide and a complete light chain antibody polypeptide may be expressed in any of the yeast described herein (e.g., Yarrowia lipolytica). As another example, that portion of a heavy chain antibody polypeptide that is included in a Fab fragment, an Fv fragment, or a scFv fragment may be expressed in a yeast along with that portion of a light chain antibody polypeptide that is included in a Fab fragment, an Fv fragment, or a scFv fragment. As will be understood by those of ordinary skill in the art, when a heavy chain antibody polypeptide and a light chain antibody polypeptide (or antibody polypeptide fragments thereof) are expressed on the surface of a yeast cell, such antibody polypeptides or fragments can associate with one another to reconstitute a functional antigen-binding molecule.

In certain embodiments, a heavy chain antibody polypeptide or antibody polypeptide fragment is expressed on the surface of a first haploid yeast cell of a first mating type, a light chain antibody polypeptide or antibody polypeptide fragment is expressed on the surface of a second haploid yeast cell of a second mating type, and the first and second haploid yeast cells are mated to produce a diploid yeast cell. Conversely, a light chain antibody polypeptide or fragment thereof is expressed on the surface of a first haploid yeast cell of a first mating type, a heavy chain antibody polypeptide or fragment thereof is expressed on the surface of a second haploid yeast cell of a second mating type, and the first and second haploid yeast cells are mated to produce a diploid yeast cell. Such diploid yeast cells produced as a result of such matings will express the heavy chain antibody polypeptide and the light chain antibody polypeptide (or antibody polypeptide fragments thereof). Yeast mating types are known in the art. For example, in haploid form, S. cerevisiae exists in one of two mating types: MATA and MATB. Moreover, MATA mating type Yarrowia lipolytica cells can be engineered to the MATB mating type. Haploid MATA and MATB yeast cells can mate with one another to form a diploid yeast cell. Those of ordinary skill in the art will be aware of yeast species that can be mated, and will also be aware of suitable mating types.

In certain embodiments, a haploid yeast cell expressing an antibody polypeptide or antibody polypeptide fragment can be generated by transforming the haploid yeast cell with a vector or expression cassette (see section entitled “Expression Cassettes and Vectors”) comprising a nucleic acid sequence that encodes the antibody polypeptide or antibody polypeptide fragment. Alternatively, a haploid yeast cell expressing an antibody polypeptide or fragment thereof can be generated by transforming a diploid yeast cell with a vector comprising a nucleic acid sequence that encodes the antibody polypeptide or fragment thereof, and sporulating the transformed diploid yeast cell to produce a haploid yeast cell.

In certain embodiments, both a heavy chain antibody polypeptide or antibody polypeptide fragment and a light chain antibody polypeptide or antibody polypeptide fragment are expressed on the surface of a yeast cell by transforming the haploid yeast cell with two vectors or expression cassettes: a first vector or expression cassette that comprises a nucleic acid sequence that encodes the heavy chain antibody polypeptide or antibody polypeptide fragment, and a second vector or expression cassette that comprises a nucleic acid sequence that encodes the light chain antibody polypeptide or antibody polypeptide fragment. In certain embodiments, both a heavy chain antibody polypeptide or antibody polypeptide fragment and a light chain antibody polypeptide or antibody polypeptide fragment are expressed on the surface of a yeast cell by transforming the haploid yeast cell with a single vector, which vector comprises expression cassettes that comprises a nucleic acid sequences that encode the heavy chain antibody polypeptide or antibody polypeptide fragment and the light chain antibody polypeptide or antibody polypeptide fragment. Such yeast cells can be either haploid or diploid.

In certain embodiment, a heavy chain antibody polypeptide or antibody polypeptide fragment and/or a light chain antibody polypeptide or antibody polypeptide fragment to be expressed on the surface of a yeast cell is a fusion polypeptide that comprises an anchor polypeptide (see section entitled “Anchor Polypeptides” below). Although anchoring an antibody polypeptide or fragment through its heavy chain antibody polypeptide or fragment is typical, anchoring via the light chain antibody polypeptide or fragment is also possible. See e.g., Lin et al., App. Microbiol. Biotechol, 2003, August; 62(2-3): 226-32, incorporated herein by reference in its entirety. In certain embodiments, only the heavy chain of an antibody polypeptide or fragment thereof is fused to an anchor polypeptide. In certain embodiments, only the light chain of an antibody polypeptide or fragment thereof is fused to an anchor polypeptide. In certain embodiments, both a heavy chain of an antibody polypeptide or fragment thereof and a light chain of an antibody polypeptide or fragment thereof are fused to an anchor polypeptide. In certain embodiments, an anchor polypeptide is fused at the amino end of the fusion polypeptide. In certain embodiments, an anchor polypeptide is fused at the carboxy end of the fusion polypeptide.

In certain embodiments, an antibody polypeptide or antibody polypeptide fragment is obtained by any of the variety of methods disclosed herein. Such an antibody polypeptide or fragment thereof may be obtained as part of the cell. Alternatively, an antibody polypeptide or fragment thereof may be purified from the cell after it is expressed. Standard techniques for purifying polypeptides may be used.

Yeast cells that express a polypeptide of interest can be detected and screened by any of a variety of methods known to those of ordinary skill in the art. For example, FACS (fluorescence-activated cell sorting) can be employed. In FACS, yeast cells are contacted with a labeled agent that binds the polypeptide of interest (e.g., an antigen that is bound by antibody polypeptides or antibody polypeptide fragments of the present disclosure). Any label can be used, so long as it is dectable. Suitable labels include, without limitation fluorescent moieties, chemiluminescent moieties, and the like. Those of ordinary skill in the art will be aware of suitable labels. In certain embodiments, an agent is labeled with an indirect label that can be detected by binding a detectably-labeled agent (e.g., a fluorescent or chemiluminescent moiety) that binds the indirect label. A variety of indirect labels are known in the art including, but not limited to, biotin (which can be bound by avidin or streptavidin), epitope tags (e.g. any of the epitope tags described herein), etc. Epitope tags can be detected using labeled antibodies of fragments thereof specific for the particular epitope tag. Alternatively, epitope tags can be detected by binding a first antibody or fragment thereof specific to the particular epitope tag, and detecting the first antibody or fragment with a labeled second antibody or fragment thereof. The yeast cells are then passed through a cell sorter that separates the cells and determines whether the labeled agent has associated with each individual cell. Those cells that exhibit fluorescence express the polypeptide of interest on their surfaces. Alternatively, cells may be “panned” on plates coated with an agent that binds the antibody polypeptide or fragment of interest (e.g. an antigen). Alternatively, cells may be bound to a solid support (e.g. a bead) that is linked to an agent that binds the antibody polypeptide or fragment of interest (e.g. an antigen). The solid support can then be isolated (e.g., by centrifugation, magnetic removal if the support is paramagnetic, etc.); any cells bound to the solid support express the polypeptide of interest on their surfaces. Those of ordinary skill in the art will be aware of other suitable methods for identifying and isolating yeast cells that express a polypeptide of interest on their surfaces. See e.g., Yeung and Wittrup, Biotechnol. Prog., March-April;18(2):212-20, 2002; Ackerman et al., Biotechnol. Prog., May-June;25(3):774-83, 2009; Wang et al., J. Immunol. Methods, September; 304(1-2):30-42, 2005; and Chao et al., Nat. Protoc., 1(2):755-68, 2006, each of which is incorprated herein by reference in its entirety.

Those of ordinary skill in the art will be aware of other antibody polypeptides and fragments that can be expressed on the surface of a yeast (e.g., Yarrowia lipolytica) cell in accordance with methods and compositions described herein.

### Anchor Polypeptides

Any of a variety of anchor polypeptides can be used to express a polypeptide (e.g., an antibody polypeptide or antibody polypeptide fragment) on the surface of a yeast cell in accordance with methods and compositions described herein.

“Anchor polypeptide” as the term is used herein refers to a polypeptide that is tethered to the surface of a cell and that can thus be used to tether other polypeptides (e.g., an antibody polypeptide or antibody polypeptide fragment) to the surface of a cell. For example, an anchor polypeptide may be a transmembrane or a cell wall protein, such as for example, a glycosylphosphatidylinositol (GPI) cell wall protein. A variety of anchor polypeptides are known in the art and can be used in accordance with the compositions and methods disclosed herein for expressing a polypeptide on the surface of a yeast. Such anchor peptides include, but are not limited to, the S. cerevisiae Aga1-Aga2 (mating type A agglutinin gene) heterodimer, S. cerevisiae alpha-agglutinin (Sag1p), Pir1p, Pir2p, Pir4p, Flo1p, Yarrowia CWPI, and fragments thereof (see e.g., Ueda et al., J. Biosci. Bioeng. 90: 125-36, 2000; Abe, H., Shimma et al., Pir. Glycobiology 13, 87-95, 2003; Andres, I., et al., Biotechnol Bioeng 89, 690-7, 2005; Wang, Q., et al., Curr. Microbiol. 56, 352-7, 2008; Tamino, T., et al., Biotechnol. Prog. 22, 989-93, 2006; Yue et al., J. Microbiol. Methods. 2008 February; 72(2):116-23; each of which is incorporated herein by reference in its entirety).

In certain embodiments, an anchor polypeptide is used to tether a polypeptide of interest (e.g., an antibody polypeptide or antibody polypeptide fragment) to the surface of a yeast cell, e.g., to the surface of a Yarrowia lipolytica cell. For example, an anchor polypeptide may be fused to the polypeptide of interest, such that both the anchor polypeptide and the polypeptide of interest are expressed on the cell surface.

In certain embodiments, yeast cell that expresses a polypeptide of interest (e.g., an antibody polypeptide or antibody polypeptide fragment) can be generated by transforming the yeast cell with a vector or expression cassette (see section entitled “Expression Cassettes and Vectors”) comprising a first nucleic acid sequence that encodes the polypeptide of interest fused in frame to a second nucleic acid sequence encoding an anchor polypeptide. In certain embodiments, the first nucleic acid sequence is fused 5′ to the second nucleic acid sequence, such that a fusion polypeptide produced from the fusion sequence comprises an N-terminal polypeptide of interest and a C-terminal anchor polypeptide. In certain embodiments, the first nucleic acid sequence is fused 3′ to the second nucleic acid sequence, such that a fusion polypeptide produced from the fusion sequence comprises an N-terminal anchor polypeptide and a C-terminal polypeptide of interest. In certain embodiments, the first nucleic acid sequence is fused directly in frame to the second nucleic acid sequence. In certain embodiments, the first nucleic acid sequence is fused to a linker sequence, which linker sequence is fused to the second nucleic acid sequence. As described in more detail in the section entitled “Expression Cassettes and Vectors”, a linker sequence typically encodes a linker polypeptide such as, without limitation, a GlySer linker polypeptide, e.g., (Gly4Ser)₃ or (GlySer)₅.

### Expression Cassettes and Vectors

In certain embodiments, a polypeptide (e.g., an antibody polypeptide or antibody polypeptide fragment) is expressed on the surface of a yeast cell by transforming the yeast with an expression cassette comprising a nucleic acid sequence encoding the polypeptide. The term “expression cassette” as used herein refers to a nucleic acid sequence that minimally comprises: (1) a nucleotide sequence encoding a polypeptide of interest, and (2) a nucleotide sequence that drives expression of the polypeptide of interest (e.g., a promoter).

In certain embodiments, a polypeptide of interest that is encoded by a nucleotide sequence of the expression cassette comprises an antibody polypeptide or antibody polypeptide fragment. An expression cassette may comprise a nucleotide sequence encoding any antibody polypeptide or fragment described herein, e.g., an antibody polypeptide or fragment derived from a Fab fragment, a Fv fragment, or a scFv fragment. In certain embodiments, a polypeptide of interest is a heavy chain of a Fab fragment. In certain embodiments, a polypeptide of interest is a light chain of a Fab fragment.

In certain embodiments, a polypeptide of interest that is encoded by a nucleotide sequence of the expression cassette comprises an anchor polypeptide. An expression cassette may comprise a nucleotide sequence encoding any anchor polypeptide described herein, e.g., the S. cerevisiae Aga1-Aga2 heterodimer, S. cerevisiae alpha agglutinin (Sag1p), Pir1p, Pir2p, Pir4p, Flo1p, Yarrowia CWPI, and fragments thereof.

In certain embodiments, a polypeptide of interest that is encoded by a nucleotide sequence of the expression cassette comprises an antibody polypeptide or antibody polypeptide fragment fused in frame to an anchor polypeptide. For example, an expression cassette can comprise a first nucleotide sequence encoding an antibody polypeptide or fragment, which first nucleotide sequence is fused in frame to a second nucleotide sequence encoding an anchor polypeptide. In certain embodiments, a first nucleotide sequence encoding an antibody polypeptide or fragment is fused in frame 5′ to a second nucleotide sequence encoding an anchor polypeptide, such that when the nucleotide sequences are expressed, the antibody polypeptide or fragment is N-terminal to the anchor polypeptide. In certain embodiments, a first nucleotide sequence encoding an antibody polypeptide or fragment is fused in frame 3′ to a second nucleotide sequence encoding an anchor polypeptide, such that when the nucleotide sequences are expressed, the antibody polypeptide or fragment is C-terminal to the anchor polypeptide.

In certain embodiments, an expression cassette comprises a nucleotide sequence encoding an antibody polypeptide or antibody polypeptide fragment is fused in frame to a nucleotide sequence encoding an anchor polypeptide, such that there are no intervening nucleotide residues. In such embodiments, the polypeptide expressed from the expression cassette will comprise the antibody polypeptide or fragment fused directly to the anchor polypeptide, with no intervening amino acid residues. In certain embodiments, an expression cassette comprises a nucleotide sequence encoding an antibody polypeptide or antibody polypeptide fragment is fused in frame to linker sequence encoding a linker polypeptide, which linker sequence is fused in frame to a nucleotide sequence encoding an anchor polypeptide, such that the linker sequence is fused in frame between the first and nucleotide sequence encoding the antibody polypeptide or fragment and the nucleotide sequence encoding the anchor polypeptide. In such embodiments, the polypeptide expressed from the expression cassette will comprise the antibody polypeptide or antibody polypeptide fragment, the linker polypeptide, and the anchor polypeptide. In any of the embodiments described in this paragraph, the nucleotide sequence encoding an antibody polypeptide or fragment may be fused either 5′ or 3′ to the nucleotide sequence encoding an anchor polypeptide.

Any of a variety of linker polypeptides may be used in accordance with the presently described compositions and methods. A linker polypeptide serves as a spacer between two polypeptides of interest that are included within a fusion polypeptide. A linker polypeptide advantageously does not interfere with the functions of the two polypeptides or interest, or interferes only to a minor extent. In certain embodiments, a linker polypeptide permits the two polypeptides of interest significant conformational freedom, such that the two polypeptides of interest are able to adopt a variety of spatial positions and orientations relative to each other. A non-limiting example of a linker polypeptides is a GlySer linker polypeptide, e.g., (Gly4Ser)₃ (SEQ ID NO:14) or (GlySer)₅ (SEQ ID NO:15). In certain embodiments, a linker polypeptide can be situated between two portions of an antibody polypeptide or antibody polypeptide fragment. For example, a linker sequence encoding a linker polypeptide can be fused in frame between 1) a heavy chain nucleic acid sequence encoding a heavy chain variable region of a scFv fragment and, 2) a light chain nucleic acid sequence encoding a light chain variable region of a scFv fragment. In certain embodiments, a polypeptide of interest includes more than one linker sequence. For example, a fusion polypeptide can comprise 1) a scFv antibody polypeptide fragment can comprises a first linker polypeptide between the heavy and light chain variable region polypeptide of the scFv fragment, 2) an anchor polypeptide, and 3) a second linker polypeptide between the scFv antibody polypeptide and the anchor polypeptide. Those of ordinary skill in the art will be aware of other suitable linker polypeptides and the nucleotide sequences encoding them.

In certain embodiments, an expression cassette comprises a leader nucleic acid sequence comprising a nucleotide sequence encoding a leader polypeptide. Any of a variety of leader polypeptides may be used in accordance with the presently described compositions and methods. A leader polypeptide functions to help drive processing of a polypeptide through the secretion apparatus, ultimately resulting in a properly processed surface displayed polypeptide. Leader sequences are cleaved from the polypeptide during processing and are not part of the fully-processed polypeptide. As will be understood by those of ordinary skill in the art, a leader nucleic acid sequence will typically be fused in frame 5′ to the nucleotide sequence encoding a polypeptide of interest, such that the leader polypeptide is at the N-terminus of the expressed fusion polypeptide. Non-limiting examples of leader polypeptides include LIP2 pre, LIP2 prepro, XPR2 pre, and XPR2 prepro. See e.g., Pignède et al., J. Bacteriol., May; 182(10):2802-10, 2000; Davidow et al., J. Bacteriol., October; 169(10):4621-9, 1987; and Madzak et al., J. Biotechnol., April 8; 109(1-2):63-81, 2004, each of which is incorporated herein by reference in its entirety. Those of ordinary skill in the art will be aware of other suitable leader polypeptides and the nucleotide sequences encoding them.

In certain embodiments, an expression cassette comprises an epitope nucleic acid sequence comprising a nucleotide sequence encoding an epitope tag. Any of a variety of epitope tags may be used in accordance with the presently described compositions and methods. An epitope tag is typically a short polypeptide sequence that facilitates detection, measurement, quantitation, and/or purification (or isolation) of an expressed polypeptide. An epitope tag may be located anywhere within a given polypeptide, e.g., at the N-terminus, at the C-terminus, or internally. Non-limiting examples of epitope tags include c-Myc (myelocytomatosis cellular oncogene), V5 (derived from the C-terminal sequence of the P and V proteins of Simian Virus 5), polyhistidine (e.g., 6-his, or hexahistidine), glutathione-5-transferase, streptavidin, biotin, hemagglutinin, Flag-tag (FLAG octapeptide), and E-tag [GAPVPYPDPLEPR, SEQ ID NO: 13]. Those of ordinary skill in the art will be aware of other suitable epitope tags and the nucleotide sequences encoding them.

In certain embodiments, an expression cassette comprises a promoter. A promoter, as is known in the art, is a nucleotide sequence that drives transcription of a downstream nucleotide sequence into ribonucleic acid (RNA), which transcription is mediated via any of a variety of transcription factors. In certain embodiments, the transcribed RNA encodes a polypeptide of interest. In certain embodiments, an expression cassette comprises a promoter operably linked to a fusion sequence comprising: (1) a first nucleic acid sequence comprising a nucleotide sequence encoding an antibody polypeptide or antibody polypeptide fragment, fused in frame to (2) a second nucleic acid sequence comprising a nucleic acid sequence comprising a nucleotide sequence encoding an anchor polypeptide.

Advantageous promoters are those that typically function in the cell of interest. For example, a number of promoters are known that function in yeast, e.g., in a Yarrowia species such as, without limitation, Yarrowia lipolytica. In certain embodiments, a promoter that functions in Yarrowia lipolytica is used to drive expression of RNA encoding an antibody polypeptide or an antibody polypeptide fragment. In certain embodiments, a promoter that functions in Yarrowia lipolytica is used to drive expression of RNA encoding an anchor polypeptide. In certain embodiments, a promoter that functions in Yarrowia lipolytica is used to drive expression of RNA encoding an antibody polypeptide or an antibody polypeptide fragment fused to an anchor polypeptide.

Any of a variety of promoters can be used in accordance with the presently described compositions and methods to express a polypeptide of interest on the surface of a yeast cell. In certain embodiments, a promoter used to express a polypeptide (e.g., an antibody polypeptide or antibody polypeptide fragment) is constitutive. A number of constitutive promoters are known in the art, including without limitation, TEFL and the glyceraldehyce-3-phosphate dehydrogenase promoter. In certain embodiments a promoter used to express a polypeptide (e.g., an antibody polypeptide or antibody polypeptide fragment) is inducible. Inducible promoters are useful when the practitioner desires to control when a polypeptide of interest is expressed. A number of inducible promoters are known in the art, including without limitation, POX3 and LIP2 promoters. In certain embodiments a promoter used to express a polypeptide (e.g., an antibody polypeptide or antibody polypeptide fragment) is semi-constitutive. A “semi-constitutive promoter” as the term is used herein refers to a promoter that is not completely constitutive and that drives expression of certain genes largely or only under certain conditions. For example, a semi-constitutive promoter may drive gene expression in a growth-phase-dependent manner. A number of semi-constitutive promoters are known in the art, including without limitation, the hp4d promoter. Those of ordinary skill in the art will be aware of suitable constitutive, inducible, and semi-constitutive promoters that function in a cell of interest, e.g., in a Yarrowia species such as, without limitation, Yarrowia lipolytica.

In certain embodiments, a polypeptide (e.g., an antibody polypeptide or antibody polypeptide fragment) is expressed on the surface of a yeast cell by transforming the yeast with a vector comprising an expression cassette, e.g., any of the expression cassettes described herein. A “Vector” as the term is used herein refers to a nucleic acid that comprises an expression cassette, and further includes one or more additional elements. In certain embodiments, a vector comprises an element that facilitates replication, homologous or non-homologous integration, and/or maintenance of the vector under selection conditions.

Any of a variety of vectors can be used in accordance with the presently described compositions and methods to express a polypeptide of interest on the surface of a yeast cell. Non-limiting examples of vectors that can be used include those disclosed in US Patent Publication No. 2008-0171359, incorporated herein by reference in its entirety. Those of ordinary skill in the art will be aware of other suitable vectors for use in a given cell (e.g., yeast cell) of interest. Moreover, any of a variety of vectors can be modified for use in expressing a polypeptide of interest on the surface of a yeast cell. For example, a commercially available or other vector may be suitable for use in a given yeast species, but such vector may not include an expression cassette that includes a promoter operably linked to a fusion sequence comprising: (1) a first nucleic acid sequence comprising a nucleotide sequence encoding an antibody polypeptide or antibody polypeptide fragment, fused in frame to (2) a second nucleic acid sequence comprising a nucleic acid sequence comprising a nucleotide sequence encoding an anchor polypeptide. Such a vector may be modified to include the promoter and nucleic acid sequences encoding the antibody polypeptide or antibody polypeptide fragment and anchor polypeptide. A number of molecular techniques are suitable for modifying vectors, many of which can be found in Sambrook, J., Fritsch, E. F., and Maniatis, T. Molecular Cloning: A Laboratory Manual. 2nd ed., Cold Spring Harbor Laboratory, Cold Spring Harbor Laboratory Press, Cold Spring Harbor, N.Y., 1989, the contents of which are incorporated herein by reference in their entirety. Those of ordinary skill in the art will be aware of a variety of other suitable molecular techniques for modifying vectors for use in expressing a polypeptide of interest on the surface of a yeast (e.g., Yarrowia lipolytica) cell.

In certain embodiments, a vector comprises a nucleotide sequence encoding a selectable marker. A “selectable marker” as the term is used herein refers to a polypeptide that permits a cell containing the selectable marker to survive and/or proliferate under conditions wherein a cell that lacks the selectable markers fails to survive and/or proliferate. The term and concept of a selectable marker are well known to those of ordinary skill in the art. Non-limiting examples of selectable markers include those for leucine (e.g., LEU2), uracil (e.g., URA3d1), adenine (e.g., ADE2), lysine (Lys), arginine (Arg), glycerol utilization (Gut), tryptophan (Trp), glycerol-3-phosphate dehydrogenase (G3p), and hygromycin B phosphotransferase (hph). Those of ordinary skill in the art will be aware of other suitable markers that can be used in accordance with the compositions and methods disclosed herein.

In certain embodiments, a vector is integrated into the genome of a cell (e.g., a Yarrowia cell such as Yarrowia lipolytica). Various techniques for integrating a vector into a cell's genome are known in the art. In certain embodiments, a vector comprises a zeta element. A zeta element is a sequence that permits a vector to integrate by homologous recombination into the genome of a Y. lipolytica strain carrying a Ylt1 retrotransposon, or by non-homologous recombination in yeast that lack the Ylt1 retrotransposon. In certain embodiments, a zeta element comprises a long terminal repeat of a retrotransposon, such as without limitation, a Ylt1 or Tyl6 retrotransposon. Those of ordinary skill in the art will be aware of other elements, and will be able to use them in vectors in accordance with the compositions and methods disclosed herein.

In certain embodiments, vector is not integrated into the genome of a cell. For example, a replicative vector may be introduced, e.g., by transformation, into a yeast cell. Replicative vectors contain suitable elements for maintenance, replication and/or other functions in a host cell. For example, a vector may contain one or more autosomal replication elements. Non-limiting examples of such autosomal replication elements include a centromere (CEN) and an origin of replication (ORI). In certain embodiments, a centromere comprises CEN1 or CEN3 (Vernis, L., et al., Mol. Cell. Biol. 17, 1995-2004, 2007, incorporated herein by reference in its entirety). In certain embodiments, an origin of replication comprises ORI1068 or ORI3018. (Fournier et al., Yeast, January; 7(1):25-36, 1991, incorporated herein by reference in its entirety). In certain embodiments, a vector that is not integrated into the genome of a cell may contain an autonomously replicating sequence (ARS). See e.g., Fournier, et al., Yeast 7, 25-36, 1991 and Matsuoka et al., Mol. Gen. Genet. 237, 327-333, 1993, each of which is incorporated herein by reference in its entirety). In certain embodiments, an ARS comprises a centromere and an origin of replication. Non-limiting examples of ARSs include ARS18 and ARS18.

In certain embodiments, an expression cassette comprises a promoter operably linked to an anchor nucleotide sequence nucleic acid sequence comprising a nucleotide sequence encoding an anchor polypeptide, wherein the anchor nucleic acid sequence can be expressed as a first fusion partner in a fusion protein comprising a second fusion partner of interest. In certain of such embodiments, an expression cassette comprises another nucleic acid sequence comprising a nucleotide sequence encoding the second fusion partner of interest. The second fusion partner of interest can be any of a variety of polypeptides. For example, the second fusion partner of interest may be an antibody polypeptide or antibody polypeptide fragment, although second fusion partners are not limited to such antibody polypeptides or fragments. Since the second fusion partner will be fused to an anchor polypeptide, the second fusion partner will also be expressed on the surface of the cell. In certain embodiments, an expression cassette embodied in this paragraph comprises a nucleic acid sequence comprising a restriction site for ease of fusing the second fusion partner of interest. Any of a variety of restriction sites can be included in an expression cassette. Those of ordinary skill in the art will be aware of suitable restriction sites and will be able to engineer expression cassettes comprising them.

In certain embodiments, a nucleotide sequence encoding a polypeptide of interest is codon optimized for use in the organisms (e.g., yeast cell) in which the polypeptide is expressed. Codon optimization is a process by which a nucleotide sequence that encodes a polypeptide of interest is modified such that the nucleotide sequence is optimized for expression in a particular organism, but the amino acid sequence of the polypeptide remains the same. A codon is a three-nucleotide sequence that is translated by a cell into a given amino acid. Since there are twenty naturally encoded amino acids, but there are sixty-four possible combinations of three-nucleotide sequences, most amino acids are coded for by multiple codons. Certain codons in given species are often translated better than other codons that encode the same amino acid, and each species differs in its codon preference. As such, a gene from one species may be poorly expressed when introduced into another species. Once way to overcome this problem is to take advantage of the degeneracy of the genetic code, and modify a nucleotide sequence that encodes a polypeptide of interest such that the nucleotide sequence now contains codons that are efficiently used in the species of interest, but which nucleotide sequence still encodes the same polypeptide. It is possible to determine which codons are the most widely used in the organism of interest. Indeed, this has already been done for a variety of organisms, including Yarrowia lipolytica. A sample codon optimization chart for Y. lipolytica based on 2,945,919 codons is shown below in Table 1. Those of ordinary skill in the art will be aware of and will be able to determine codon usage for other organisms.

TABLE 1
Yarrowia lipolytica Codon Usage Table

| | | | |
|---|---|---|---|
| UUU 15.9(46804) | CU 21.8(64161) | AU 6.8(20043) | GU 6.1(17849) |
| UUC 23.0(67672) | CC 20.6(60695) | AC 23.1(68146) | GC 6.1(17903) |
| UUA 1.8(5280) | CA 7.8(22845) | AA 0.8(2494) | GA 0.4(1148) |
| UUG 10.4(30576) | CG 15.4(45255) | AG 0.8(2325) | GG 12.1(35555) |
| CUU 13.2(38890) | CU 17.4(51329) | AU 9.6(28191) | GU 6.0(17622) |
| CUC 22.6(66461) | CC 23.3(68633) | AC 14.4(42490) | GC 4.4(12915) |
| CUA 5.3(15548) | CA 6.9(20234) | AA 9.8(28769) | GA 21.7(63881) |
| CUG 33.5(98823) | CG 6.8(20042) | AG 32.1(94609) | GG 7.7(22606) |
| AUU 22.4(66134) | CU 16.2(47842) | AU 8.9(26184) | GU 6.7(19861) |
| AUC 24.4(71810) | CC 25.6(75551) | AC 31.3(92161) | GC 9.8(28855) |
| AUA 2.2(6342) | CA 10.5(30844) | AA 12.4(36672) | GA 8.4(24674) |
| AUG 22.6(66620) | CG 8.5(25021) | AG 46.5(136914) | GG 2.4(7208) |
| GUU 15.8(46530) | CU 25.5(75193) | AU 21.5(63259) | GU 16.6(48902) |
| GUC 21.5(63401) | CC 32.7(96219) | AC 38.3(112759) | GC 21.8(64272) |
| GUA 4.0(11840) | CA 11.2(32999) | AA 18.8(55382) | GA 20.9(61597) |
| GUG 25.7(75765) | CG 8.9(26190) | AG 46.2(136241) | GG 4.4(12883) |

Legend: Table fields are shown as [triplet] [frequency: per thousand] ([number]).
Data was derived from 2,945,919 codons present in 5,967 coding sequences.
Table contents obtained from Codon Usage Database found and can be found at the URL www.kazusa.or.jp/codon/cgi-bin/showcodon.cgi?species=284591.

In certain embodiments, vectors or expression cassettes comprising one or more of the Yarrowia lipolytica codon-optimized nucleic acid sequences of SEQ ID NOs: 1-12, shown below, can be transformed into Yarrowia lipolytica for expression. The relevant coding sequences within each of the codon-optimized nucleic acid sequences below are indicated by bold, underlined text.

SEQ ID NO: 1: Synthetic Yarrowia lipolytica codon optimized C-terminal S.
cerevisiae SAG1p (320 C-terminal amino acids) (SfiI/NotI flanked)
[gaatgcagcggcccagccggccatggcccaggtgcagctgcaggtcgacctcgagtggcggcggaggctctggcggaggcggatct
ggcggcggtggcagtgcacaggtccaactgcaggagctcgatatcaaacgggcggccgcagagcagaagctgatctctgaggaagatc
tgtccggcggaggcggctccggtggcggcggttctggcggtggcggctctcatatgtctgccaagtcctctttcatctctaccaccaccacc
gacctgacctctatcaacacctctgcctactctaccggctctatctctaccgtggagaccggcaaccgaaccacctctgaagtgatctctcac
gtggtgaccacttctaccaagctgtctcccaccgccaccacctccctgaccattgcccagacctctatctactccaccgactccaacatcacc
gtgggcaccgacatccacaccacctccgaggtcatttccgacgtggagaccatctcccgagagaccgcctctaccgtggtggccgctcct
acctctaccaccggctggaccggcgccatgaacacctacatctctcagttcacctcttcttccttcgccaccatcaactctacccccatcatctc
ttcctctgccgtgttcgagacctctgacgcctctatcgtgaacgtccacaccgagaacattaccaacaccgccgctgttccctctgaggaacc
cacctttgtgaacgccacccgaaactccctgaactctttctgttcttctaagcagccctcctctccctcttcctacacctcttcccccctggtgtcc
tctctgtctgtgtctaagaccctgctgtctacctctttcaccccctctgtgcccacctctaacacctacattaagaccaagaacaccggctacttc
gagcacaccgccctgaccacctcttctgtgggcctgaactccttctctgagaccgccgtgtcctctcagggcaccaagatcgacacctttctg
gtctcctccctgatcgcctacccctcttctgcctctggctctcagctgtctggcatccagcagaacttcacctctacctccctgatgatctctacc
tacgagggcaaggcctctatcttcttctctgccgagctgggctctatcatcttcctgctgctgtcttacctgctgttctaacctagg]
SEQ ID NO: 2: Synthetic Yarrowia lipolytica codon optimized C-terminal S.
cerevisiae AGA2p (SfiI/NotI flanked)
[gaatgcagcggcccagccggccatggcccaggtgcagctgcaggtcgacctcgagtggcggcggaggctctggcggaggcggatct
ggcggcggtggcagtgcacaggtccaactgcaggagctcgatatcaaacgggcggccgcagagcagaagctgatctctgaggaagatc
tgtccggcggaggcggctccggtggcggcggttctggcggtggcggctctcatatgcaggaactgaccaccatctgcgagcagattccct
ctcccaccctggagtctaccccctactctctgtctaccaccaccatcctggccaacggcaaggccatgcagggcgtgttcgagtactacaag
tctgtgaccttcgtgtctaactgtggctctcacccctctaccacctctaagggctctcccatcaacacccagtacgtgttctaacctagg]
SEQ ID NO: 3: Synthetic Yarrowia lipolytica codon optimized C-terminal
Yarrowia lipolytica CWPI (SfiI/NotI flanked)
[gaatgcagcggcccagccggccatggcccaggtgcagctgcaggtcgacctcgagtggcggcggaggctctggcggaggcggatct
ggcggcggtggcagtgcacaggtccaactgcaggagctcgatatcaaacgggcggccgcagagcagaagctgatctctgaggaagatc
tgtccggcggaggcggctccggtggcggcggttctggcggtggcggctctcatatgggcaacggttacgccgtcgacgacaactccaag
tgcgaggacgacggaatccccttcggcgcctacgctgttgctgacacctccgcagagtcttctgccgcccccgcctcttctgccgccgctg
ccgagtcctctgccgccccctcttccgctgctgaggccaagcccaccgctggaggtaacaccggcgccgtcgtcacccagatcggtgac
ggccagatccaggctcccccctctgctcctcccgctgcccccgagcaggccaacggcgccgtctctgtcggtgtttctgccgccgctctcg
gtgtcgctgccgccgctctcctcatttaacctagg]
SEQ ID NO: 4: Synthetic Yarrowia lipolytica codon optimized N-terminal
Yarrowia lipolytica AGA2 (SfiI/NotI flanked)
[gaatgcacaggaactgaccaccatctgcgagcagattccctctcccaccctggagtctaccccctactctctgtctaccaccaccatcctgg
ccaacggcaaggccatgcagggcgtgttcgagtactacaagtctgtgaccttcgtgtctaactgtggctctcacccctctaccacctctaagg
gctctcccatcaacacccagtacgtgttctcttctggcggcggaggctctggcggaggcggatctggtggcggaggatctgcggcccagc
cggccatggcccaggtgcagctgcaggtcgacctcgagtggaggcggcggatctggcggtggcggctccggcggtggaggcagtgca
caggtccaactgcaggagctcgatatcaaacgggcggccgcagagcagaagctgatctctgaggaagatctgcgaaccggccaccacc
accaccaccactaacctagg]
SEQ ID NO: 5: Synthetic Yarrowia lipolytica codon optimized Herceptin scFv
(SfiI/NotI flanked)
[ggcccagccggccgaggtgcagctggtcgagtctggcggcggactggtgcagcccggtggctctctgcgactgtcttgtgccgcctctg
gcttcaacatcaaggacacctacatccactgggtgcgacaggctcccggaaagggcctggagtgggtggcccgaatctaccccaccaac
ggctacacccgatacgccgactctgtgaagggccgattcaccatctctgccgacacctctaagaacaccgcctacctgcagatgaactctct
gcgagccgaggacaccgctgtgtactactgttctcgatggggaggcgacggcttctacgccatggactactggggccagggcaccctggt
gaccgtgtcctctggcggaggcggctccggcggaggcggatctggtggcggaggctctgacatccagatgacccagtctccctcttctct
gtctgcctctgtgggcgaccgagtgaccatcacctgtcgagcctctcaggacgtgaacaccgccgtggcctggtatcagcagaagcccgg
caaggcccccaagctgctgatctactctgcctctttcctgtactctggcgtgccctctcgattctctggctctcgatctggcaccgacttcaccct
gaccatctcttctctgcagcctgaggatttcgccacctactactgtcagcagcactacaccaccccccccaccttcggccagggaaccaagg
tggagatcaaggcggccgc]
SEQ ID NO: 6: Synthetic Yarrowia lipolytica codon optimized 4-4-20 scFv
(SfiI/NotI flanked)
[ggcccagccggccgacgtgaagctggacgagactggaggaggcctggtgcagcccggacgacccatgaagctgtcttgtgtggcctct
ggcttcaccttctctgactactggatgaactgggtgcgacagtctcccgagaagggcctggagtgggtggcccagatccgaaacaagccct
acaactacgagacctactactctgactctgtgaagggccgattcaccatgtcccgagatgactctaagtcctctgtgtacctgcagatgaaca
acctgcgagtggaggacatgggcatctactactgtaccggctcttactacggcatggactactggggccagggcacctctgtgaccgtgtc
ctctggcggcggaggctctggcggaggcggatctggtggcggaggatctgacgtggtgatgacccagacccccctgtctctgcccgtgtc
tctgggcgaccaggcctctatctcttgtcgatcttctcagtctctggtccactctaacggcaacacctacctgcgatggtatctgcagaagccc
ggccagtctcccaaggtgctgatctacaaggtgtctaaccgattctctggcgtgcccgaccgattctccggctctggctctggcaccgacttc
accctgaagatctcccgagtggaggccgaggacctgggcgtgtacttctgttctcagtctacccacgtgccctggaccttcggcggaggca
ccaagctggagatcaaggcggccgc]
SEQ ID NO: 7: Synthetic Yarrowia lipolytica codon optimized anti-HEL D1.3
scFv (SfiI/NotI flanked)
[ggcccagccggcccaggtgcagctgcaggaatctggccccggactggtggccccctctcagtctctgtctatcacctgtaccgtgtctgg
cttctctctgaccggctacggcgtgaactgggtgcgacagccccctggcaagggcctggagtggctgggcatgatctggggcgacggca
acaccgactacaactctgccctgaagtctcgactgtctatctctaaggacaactctaagtctcaggtgttcctcaagatgaactctctccacacc
gacgacaccgcccgatactactgtgcccgagagcgagactaccgactggactactggggccagggcaccaccgtgaccgtgtcctctgg
cggtggaggctctggcggaggcggatctggtggcggaggatctgacatcgagctgacccagtctcccgcctctctgtctgcctctgtgggc
gagaccgtgaccatcacctgtcgagcctctggcaacatccacaactacctggcctggtatcagcagaagcagggcaagtctccccagctg
ctggtgtactacaccaccaccctggccgacggcgtgccctctcgattctctggctctggatctggcacccagtactccctgaagatcaactcc
ctgcagcccgaggacttcggctcttactactgtcagcacttctggtctaccccccgaaccttcggcggaggcaccaagctggagatcaagc
gagcggccgc]
SEQ ID NO: 8: Synthetic Yarrowia lipolytica codon optimized anti-HEL M3
scFv (SfiI/NotI flanked)
[ggcccagccggcccaggtgcagctgcaggaatctggccccggactggtggccccctctcagtctctgtctatcacctgtaccgtgtctgg
cttctctctgaccggctacggcgtgaactgggtgcgacagctgcctggcaagggcctggagtggctgggcatgatctggggcgacggca
acaccgcctacaactctgccctgaagtctcgactgtctatctctaaggacaactctaagtctcaggtgttcctcaagatggactctctccacac
cgacgacaccgcccgatactactgtgcccgagagcgagactaccgactggactactggggccagggcaccaccgtgaccgtgtcctctg
gcggtggaggctctggcggaggcggatctggtggcggaggatctgacatcaagctgacccagtctcccgcctctctgtctgcctctgtggg
cgagaccgtgaccatcacctgtcgagcctctggcaacacccacaactacctggcctggtatcagcagaagcagggcaagtctccccagct
gctggtgtactacaccaccaccctggccgacggcgtgccctctcgattctctggctctggatctggcacccagtactccctgaagatcaactc
cctgcagcccgaggacttcggctcttactactgtcagcacttctggtctaccccccgatctttcggcggaggcaccaagctggagatcaagc
gagcggccgc]
SEQ ID NO: 9: Synthetic Yarrowia lipolytica codon optimized 4-4-20 Fab heavy
chain (SfiI/NotI flanked)
[ggcccagccggccgacgtgaagctggacgagactggaggaggcctggtgcagcccggacgacccatgaagctgtcttgtgtggcctct
ggcttcaccttctctgactactggatgaactgggtgcgacagtctcccgagaagggcctggagtgggtggcccagatccgaaacaagccct
acaactacgagacctactactctgactctgtgaagggccgattcaccatgtcccgagatgactctaagtcctctgtgtacctgcagatgaaca
acctgcgagtggaggacatgggcatctactactgtaccggctcttactacggcatggactactggggccagggcacctctgtgaccgtgtc
ctctgctagcaccaagggaccttctgtgtttcctctggccccctcttctaagtctacctctggtggaactgctgctctgggatgtctggtgaagg
actactttcctgagcctgtgactgtgtcttggaactctggcgctctgacttctggtgttcacaccttccctgctgttctgcagtcctctggactgta
ctctctctcttctgtggtgaccgtgccttcttcttctctgggaacccagacctacatctgtaacgtgaaccacaagccctctaacactaaggtgg
acaagcgagtggagcctgcggccgc]
SEQ ID NO: 10: Synthetic Yarrowia lipolytica codon optimized 4-4-20 Fab light
chain (SfiI/NotI flanked)
[ggcccagccggccgacgtggtgatgacccagacccccctgtctctgcccgtgtctctgggcgaccaggcctctatctcttgtcgatcttctc
agtctctggtccactctaacggcaacacctacctgcgatggtatctgcagaagcccggccagtctcccaaggtgctgatctacaaggtgtct
aaccgattctctggcgtgcccgaccgattctccggctctggctctggcaccgacttcaccctgaagatctcccgagtggaggccgaggacc
tgggcgtgtacttctgttctcagtctacccacgtgccctggaccttcggcggaggcaccaagctggagatcaagcgtacggtggctgctcct
tctgtgttcattttccccccctctgacgagcagctgaagtctggaactgcttctgttgtgtgcctgctgaacaacttttacccccgagaggctaa
ggttcagtggaaggtggacaacgctctgcagtctggaaactctcaggagtctgttactgagcaggactctaaggactcgacctactctctctc
ttctaccctgaccctgtctaaggctgactacgagaagcataaggtgtacgcttgtgaggttacccatcagggactgtcctctcccgtgaccaa
gtcttttaaccgaggcgagtgcgcggccgc]
SEQ ID NO: 11: Synthetic Yarrowia lipolytica codon optimized Herceptin Fab
heavy chain (SfiI/NotI flanked)
[ggcccagccggccgaggtgcagctggtcgagtctggcggcggactggtgcagcccggtggctctctgcgactgtcttgtgccgcctctg
gcttcaacatcaaggacacctacatccactgggtgcgacaggctcccggaaagggcctggagtgggtggcccgaatctaccccaccaac
ggctacacccgatacgccgactctgtgaagggccgattcaccatctctgccgacacctctaagaacaccgcctacctgcagatgaactctct
gcgagccgaggacaccgctgtgtactactgttctcgatggggaggcgacggcttctacgccatggactactggggccagggcaccctggt
gaccgtgtcctctgctagcaccaagggaccttctgtgtttcctctggccccctcttctaagtctacctctggtggaactgctgctctgggatgtct
ggtgaaggactactttcctgagcctgtgactgtgtcttggaactctggcgctctgacttctggtgttcacaccttccctgctgttctgcagtcctct
ggactgtactctctctcttctgtggtgaccgtgccttcttcttctctgggaacccagacctacatctgtaacgtgaaccacaagccctctaacact
aaggtggacaagcgagtggagcctgcggccgc]
SEQ ID NO: 12: Synthetic Yarrowia lipolytica codon optimized Herceptin Fab
light chain (SfiI/NotI flanked)
[ggcccagccggccgacatccagatgacccagtctccctcttctctgtctgcctctgtgggcgaccgagtgaccatcacctgtcgagcctct
caggacgtgaacaccgccgtggcctggtatcagcagaagcccggcaaggcccccaagctgctgatctactctgcctctttcctgtactctg
gcgtgccctctcgattctctggctctcgatctggcaccgacttcaccctgaccatctcttctctgcagcctgaggatttcgccacctactactgtc
agcagcactacaccaccccccccaccttcggccagggaaccaaggtggagatcaagcgtacggtggctgctccttctgtgttcattttcccc
ccctctgacgagcagctgaagtctggaactgcttctgttgtgtgcctgctgaacaacttttacccccgagaggctaaggttcagtggaaggtg
gacaacgctctgcagtctggaaactctcaggagtctgttactgagcaggactctaaggactcgacctactctctctcttctaccctgaccctgt
ctaaggctgactacgagaagcataaggtgtacgcttgtgaggttacccatcagggactgtcctctcccgtgaccaagtcttttaaccgaggc
gagtgc]

### Yeast

Any of a variety of yeasts can be employed in accordance with methods and compositions described herein. Yeasts are fungal eukaryotic micro-organisms. Yeasts primarily exist in unicellular form, although some species, e.g., Yarrowia species, are dimorphic, i.e., they can also exist in a unicellular or hyphal form. Moreover, some species become multicellular through the formation of a string of connected budding cells known as “pseudohyphae”.

A number of yeasts are known to those of ordinary skill in the art. Exemplary yeasts that can be used in accordance with the presently disclosed compositions and methods include, but are not limited to: Aciculoconidium aculeatum, Candida albicans, Candida albicans var. stellatoidea, Candida bentonensi, Candida catenulata, Candida curvata, Candida famata, Candida glabrata, Candida guilliermondii, Candida hispaniensis, Candid humicola, Candida intermedia, Candida kefyr, Candida krusei, Candida lipolytica, Candida loxderi, Candida macedoniensis, Candida magnoliae, Candida maltosa, Candida melinii, Candida nitratophila, Candida parapsilosis, Candida pelliculosa, Candida pintolopesii, Candida pinus, Candida pulcherrima, Candida robusta, Candida rugosa, Candida tropicalis, Candida utilis, Candida zeylanoides, Clavispora lusitaniae, Cryptococcus albidus, Cryptococcus albidus var. diffluens, Cryptococcus kuetzingii, Cryptococcus laurentii, Cryptococcus luteolus, Cryptococcus neoformans var. gattii, Cryptococcus neoformans var. neoformans, Cryptococcus terreus, Cryptococcus uniguttulatus, Debaryomyces hansenii var. hansenii, Debaryomyces polymorphus, Endomycopsis burtonii, Endomycopsis fibuligera, Filobasidium capsuligenum, Geotrichum candidum, Hansenula anomala, Hansenula capsulata, Hansenula glucozyma, Hansenula jadinii, Hansenula petersonii, Hansenula polymorphs, Hansenula wickerhamii, Kloeckera boidinii, Kluyveromyces lactis, Kluyveromyces marxianus var. lactis, Malassezia furfur, Malassezia pachydermatis, Pichia fermentans, Pichia membranaefaciens, Pichia pastoris, Pichia pinus, Pichia subpelliculosa, Rhodotorula acheniorum, Rhodotorula araucariae, Rhodotorula graminis, Rhodotorula glutinus, Rhodotorula minuta, Rhodotorula rubra, Saccharomyces cerevisiae, Saccharomyces ellipsoideus, Schizosaccharomyces japonicus, Schizosaccharomyces pombe, Sporobolomyces holsticus, Sporobolomyces roseus, Sporobolomyces salmonicolor, Torulaspora delbrueckii, Trichosporon capitatum, Trichosporon cutaneum, Trichosporon fennicum, Trichosporon fermentans, Trichosporon pullulans, Yarrowia lipolytica, and Zygosaccharomyces rouxii. Those of ordinary skill in the art will be aware of other suitable yeasts can be used in accordance with the presently disclosed compositions and methods.

In certain embodiments, a yeast species to be employed in accordance with compositions and methods for displaying antibody polypeptides or antibody polypeptide fragments disclosed herein is a yeast of the Yarrowia genus. For example, an antibody polypeptide or antibody polypeptide fragment, e.g., any of the antibody polypeptides or fragments described herein, may be displayed on the surface of a Yarrowia lipolytica yeast cell.

Yarrowia lipolytica is a commercially useful species of hemiascomycetous yeast that is known to assimilate hydrocarbons and produce citric acid from n-alkanes, vegetable oils or glucose under aerobic conditions. For example, Yarrowia lipolytica is known to degrade palm oil mill effluent, TNT, and other hydrocarbons such as alkanes, fatty acids, fats and oils. Yarrowia lipolytica is distantly related to most other yeast species, and shares a number of common properties with filamentous fungi. Yarrowia lipolytica has a haplo-diplontic cycle in that it alternates between haploid and diploid phases.

In certain embodiments, a yeast cell is transformed with a vector or expression cassette comprising a nucleotide sequence encoding a polypeptide of interest. Any of a variety of yeast transformation methods may be used in accordance with the compositions and methods disclosed herein. Non-limiting examples of transformation methods include heat shock, electroporation and lithium acetate-mediated transformation. Those of ordinary skill in the art will be aware of yeast transformation methods suitable for the yeast to be transformed.

### Growth Conditions

In certain embodiments, a yeast cell (e.g., any of the yeast cells described herein) is grown or propagated in culture. For example, a yeast cell transformed with one or more expression cassettes or vectors as described herein in the section entitled “Expression Cassettes or Vectors” may be grown or propagated in culture. In certain embodiments a yeast of the genus Yarrowia, e.g., Yarrowia lipolytica, is grown or propagated in culture. In certain embodiments, a Yarrowia cell is cultured under a Yarrowia cell operating condition. The term “Yarrowia cell operating condition” as used herein refers to a growth or culture conditions under which the Yarrowia cell exhibits improved display of a polypeptide (e.g., an antibody polypeptide or antibody polypeptide fragment) on its surface as compared to a Yarrowia cell that is not grown under that Yarrowia cell operating condition. For example, a Yarrowia cell grown under a Yarrowia cell operating condition may exhibit: increased levels of the polypeptide on its surface, improved stability, conformation or function of the expressed polypeptide, or maintenance of expression of the polypeptide for an increased length of time.

In certain embodiments, a Yarrowia cell operating condition comprises a low induction temperature. For example, a Yarrowia cell comprising a vector or expression cassette for expressing an antibody polypeptide or antibody polypeptide fragment may be grown for some or all of the cell culture at a low induction temperature. As described in Example 3 below, folding stress is generally decreased at lower cultivation temperatures. Thus, folding stress and other detrimental processes may be decreased or eliminated by growing such a Yarrowia cell under low induction temperatures. In certain embodiments, a Yarrowia cell is grown at an induction temperature range of between about 15 and about 25 degrees Celsius, e.g., between about 15 and about 24 degrees Celsius, between about 15 and about 23 degrees Celsius, between about 15 and about 22 degrees Celsius, between about 15 and about 21 degrees Celsius, between about 15 and about 20 degrees Celsius, between about 16 and about 25 degrees Celsius, between about 17 and about 25 degrees Celsius, between about 18 and about 25 degrees Celsius, between about 19 and about 25 degrees Celsius, between about 20 and about 25 degrees Celsius, and any range in between. In certain embodiments, a Yarrowia cell is grown at an induction temperature of about 15 degrees Celsius, about 16 degrees Celsius, about 17 degrees Celsius, about 18 degrees Celsius, about 19 degrees Celsius, about 20 degrees Celsius, about 21 degrees Celsius, about 22 degrees Celsius, about 23 degrees Celsius, about 24 degrees Celsius, or about 25 degrees Celsius. “About” as the term is used herein in reference to temperature refers to a range around a given temperature value. Generally, when used in reference to a given temperature value, the term “about” refers to a range of values within +/−10% of that value, e.g., +/−9% of that value, +/−8% of that value, +/−7% of that value, +/−6% of that value, 5% of that value, +/−4% of that value, +/−3% of that value, +/−2% of that value, +/−1% of that value, or less. When used in reference to a given temperature value, the term “about” encompasses the exact value, e.g., as determined within experimental error. In certain embodiments a Yarrowia cell is grown at a higher induction temperature or temperature range during one portion of the cell culture (e.g., the initial portion), but at a lower induction temperature or temperature range during a different portion of the cell culture (e.g., the final portion). In certain embodiments, a Yarrowia cell is grown at a lower induction temperature or temperature range during that portion of the cell culture when the polypeptide of interest is being expressed. For example, a nucleotide sequence encoding an antibody polypeptide or antibody polypeptide fragment may be operably linked to an inducible promoter, and the Yarrowia cell may be grown at a lower induction temperature or temperature range during that portion of the cell culture when the promoter is induced to express the antibody polypeptide or antibody polypeptide fragment.

In certain embodiments, a Yarrowia cell operating condition comprises a short induction time. For example, a Yarrowia cell comprising a vector or expression cassette for expressing an antibody polypeptide or antibody polypeptide fragment may be grown in cell culture for a short induction time. As described in Example 4 below, shorter induction times resulted in increased expression levels of antibody polypeptide fragments. In certain embodiments, a Yarrowia cell is grown for an induction time of about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or for any induction time between these values. “About” as the term is used herein in reference to an induction time value, refers to a range around a given value. Generally, when used in reference to a given induction time value, the term “about” refers to a range of values within +/−10% of that value, e.g., +/−9% of that value, +/−8% of that value, +/−7% of that value, +/−6% of that value, 5% of that value, +/−4% of that value, +/−3% of that value, +/−2% of that value, +/−1% of that value, or less. When used in reference to a given induction time value, the term “about” encompasses the exact value, e.g., as determined within experimental error.

In certain embodiments, a Yarrowia cell operating condition comprises a low pH. For example, a Yarrowia cell comprising a vector or expression cassette for expressing an antibody polypeptide or antibody polypeptide fragment may be grown for some or all of the cell culture at a low pH. As described in Example 5 below, pH is one factor that regulates the dimorphic transition of Yarrowia is the pH of the growth media; mycelium formation is maximal at pH near neutrality and decreases as pH is lowered to become almost null at pH 3. Thus, mycelium formation may be decreased or eliminated by growing a Yarrowia cell in a low pH culture. In certain embodiments, a Yarrowia cell is grown at a pH range of between about 2 and about 4, e.g., between about 2.1 and about 4, between about 2.2 and about 4, between about 2.3 and about 4, between about 2.4 and about 4, between about 2.5 and about 4, between about 2.6 and about 4, between about 2.7 and about 4, between about 2.8 and about 4, between about 2.9 and about 4, between about 3 and about 4, between about 2 and about 3.9, between about 2 and about 3.8, between about 2 and about 3.7, between about 2 and about 3.6, between about 2 and about 3.5, between about 2 and about 3.4, between about 2 and about 3.3, between about 2 and about 3.2, between about 2 and about 3.1, between about 2 and about 3, between about 2.5 and 3.5, between about 2.5 and 3, between about 3 and 3.5 or any pH range in between. In certain embodiments, a Yarrowia cell is grown at a pH of about 2, about 2.1, about 2, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, or about 4. “About” as the term is used herein in reference to pH, refers to a range around a given value. Generally, when used in reference to a given pH value, the term “about” refers to a range of values within +/−10% of that value, e.g., +/−9% of that value, +/−8% of that value, +/−7% of that value, +/−6% of that value, 5% of that value, +/−4% of that value, +/−3% of that value, +/−2% of that value, +/−1% of that value, or less. When used in reference to a given pH value, the term “about” encompasses the exact value, e.g., as determined within experimental error. In certain embodiments a Yarrowia cell is grown at a higher pH or pH range during one portion of the cell culture (e.g., the initial portion), but at a lower pH or pH range during a different portion of the cell culture (e.g., the final portion). In certain embodiments, a Yarrowia cell is grown at a lower pH or pH range during that portion of the cell culture when the polypeptide of interest is being expressed. For example, a nucleotide sequence encoding an antibody polypeptide or antibody polypeptide fragment may be operably linked to an inducible promoter, and the Yarrowia cell may be grown at a lower pH or pH range during that portion of the cell culture when the promoter is induced to express the antibody polypeptide or antibody polypeptide fragment.

In certain embodiments, a Yarrowia cell operating condition comprises high aeration. For example, a Yarrowia cell comprising a vector or expression cassette for expressing an antibody polypeptide or antibody polypeptide fragment may be grown for some or all of the cell culture under a high aeration condition. As described in Example 3 below, increasing the aeration of a cell culture improves the cell surface display of an expressed antibody polypeptide fragment. In certain embodiments, a Yarrowia cell is grown in a shake flask to improve aeration. In certain embodiments, percent oxygen saturation of the culture is measured, and is kept above a given level to ensure that the culture is grown under sufficiently high aeration conditions. For example, under fermentor conditions, a high aeration condition may be achieved at 30-50% oxygen saturation, e.g., at least 30%, 35%, 40%, 45%, 50%, 55%, 60%, 65%, 70%, 75%, 80%, 85%, 90%, 95%, or higher. Other vessels useful in improving cell culture aeration will be known to those of ordinary skill in the art.

In certain embodiments, a Yarrowia cell operating condition comprises growing the culture in minimal medium. As described in Example 3 below, incubating cell culture in minimal medium improves the cell surface display of an expressed antibody polypeptide fragment. “Minimal medium” as the term is used herein refers to a medium that comprises the minimal elements required to support growth of a cell culture (e.g., a Yarrowia cell culture). A minimal medium typically contains a carbon source for growth (e.g., glucose), various trace elements in form of salts (e.g., magnesium, nitrogen, phosphorus, and/or sulfur), a nitrogen source, and water. A minimal medium lacks yeast extract, bactopeptone, or both. A given organism may be able to grow when grown in one minimal medium, but may not be able to grow when grown in another minimal medium. In certain embodiments, a Yarrowia cell operating condition comprises growing the culture in minimal supplemented medium. “Minimal supplemented medium” as the term is used herein refers to a minimal medium that is supplemented with amino acids. A minimal supplemented medium may be supplemented with one or a few amino acids, or may be supplemented with the complete set of all twenty amino acids used by most organisms. Those of ordinary skill in the art will be aware of a variety of minimal media, and will be able to determine which minimal medium can be used to support growth of a given organism in accordance with the compositions and methods disclosed herein.

In certain embodiments, a Yarrowia cell is grown under two or more Yarrowia cell operating conditions simultaneously. For example, a Yarrowia cell is grown under two or more Yarrowia cell operating conditions selected from the group consisting of: a low induction temperature, a short induction time, a low pH, high aeration, growth in minimal medium, and combinations thereof.

It is generally reported that 60-80% of Saccharomyces cerevisiae cells transformed with a vector for surface display of a polypeptide actually express the polypeptide on their surfaces. In contrast, using methods and compositions described herein, a much higher percentage of Yarrowia cells grown under one or more Yarrowia operating conditions exhibit an antibody polypeptide or antibody polypeptide fragment on their surfaces. For example, at least about 60%, at least about 65%, at least about 70%, at least about 75%, at least about 80%, at least about 85%, at least about 90%, at least about 95%, at least about 96% at least about 97% at least about 98%, or at least about 99% of Yarrowia cells grown under one or more Yarrowia operating conditions exhibit an antibody polypeptide or antibody polypeptide fragment on their surfaces. “About” as used in reference to the number of Yarrowia cells exhibiting an antibody polypeptide or fragment on their surfaces refers to a value within 5% of that value, and also includes the exact value. In certain embodiments, more than about 99% (e.g., 100%) of Yarrowia cells grown under one or more Yarrowia operating conditions exhibit an antibody polypeptide or antibody polypeptide fragment on their surfaces.

In certain embodiments, a Yarrowia cell comprising a vector or expression cassette for expressing an antibody polypeptide or antibody polypeptide fragment further comprises a chaperone polypeptide. As is known in the art, chaperone polypeptides assist in the non-covalent folding and/or assembly of other polypeptides. As described in Example 7, overexpression of molecular chaperones such as protein disulfide isomerase (PDI) and immunoglobulin binding protein (Kar2/BiP) in S. cerevisiae and P. pastoris improved expression of scFv and Fab fragments. In yeast, BiP/GRP78 is encoded by the KAR2 gene. Thus, in certain embodiments, a Yarrowia cell is transformed with a nucleic acid comprising a nucleotide sequence encoding a chaperone polypeptide. Non-limiting examples of chaperone polypeptides that can be advantageously used in accordance with the compositions and methods disclosed herein include PDI, Kar2/Bip, and HACI. In certain embodiments, a Yarrowia cell is transformed with a nucleic acid comprising a nucleotide sequence encoding a chaperone polypeptide under control of a promoter. For example, a chaperone may be under control of a constitutive, semi-constitutive, or inducible promoter. In certain embodiments, a chaperone polypeptide is expressed during the same portion of a cell culture as the polypeptide of interest (e.g., an antibody polypeptide or antibody polypeptide fragment). Those of ordinary skill in the art will be aware of other chaperone polypeptides, and will be able to use them and assess their efficacy when used with the presently disclosed compositions and methods.

### Applications

Compositions and methods disclosed herein can be used in a variety of applications. As one non-limiting example, compositions and methods disclosed herein can be used to screen a library of antibody polypeptides or antibody polypeptide fragments for the ability to bind a given antigen.

In certain embodiments, a yeast cell (e.g., a Yarrowia cell such as Yarrowia lipolytica) displays an antibody polypeptide or antibody polypeptide fragment on its surface, and the cell is tested for its ability to bind a given antigen. In certain embodiments, a yeast cell expresses two antibody polypeptides or antibody polypeptide fragments, which antibody polypeptides or fragments thereof associate with one another such that together they are capable of binding an antigen. For example, a heavy chain Fab fragment and a light chain Fab fragment can be displayed on the cell surface of a yeast, which Fab fragments associate with one another to form a functional antigen-binding moiety. In certain embodiments, a scFv antibody polypeptide fragment is displayed on the cell surface of a yeast, which scFv fragment can bind a given antigen. In certain embodiments, a yeast cell (e.g., a Yarrowia cell such as Yarrowia lipolytica) is transformed with a vector or an expression cassette comprising a nucleic acid sequence comprising a nucleotide sequence encoding an antibody polypeptide or antibody polypeptide fragment. In certain embodiments, a yeast cell (e.g., a Yarrowia cell such as Yarrowia lipolytica) is transformed with two or more vectors and/or expression cassettes, each of which comprises a nucleic acid sequence comprising a nucleotide sequence encoding an antibody polypeptide or antibody polypeptide fragment. In certain embodiments, a yeast cell (e.g., a Yarrowia cell such as Yarrowia lipolytica) is transformed with a vector or an expression cassette comprising a two or more nucleic acid sequences, each of which comprises a nucleotide sequence encoding an antibody polypeptide or antibody polypeptide fragment.

In certain embodiments, a plurality of yeast cells (e.g., a Yarrowia cell such as Yarrowia lipolytica) is transformed with a library of vectors or expression cassettes, which library comprises a plurality of nucleic acid sequences comprising nucleotide sequences encoding a plurality of antibody polypeptides or antibody polypeptide fragments, to generate an antibody polypeptide yeast library. As used herein, the term “antibody polypeptide yeast library” refers to a plurality of yeast cells displaying a plurality of antibody polypeptides or antibody polypeptide fragments on their surface. Such an antibody polypeptide yeast library can be used to screen for antibody polypeptides or antibody polypeptide fragments in the library that bind one or more particular antigens.

In certain embodiments, a plurality of yeast cells (e.g., a Yarrowia cell such as Yarrowia lipolytica) is transformed with a library of vectors or expression cassettes, which library comprises a plurality of nucleic acid sequences comprising nucleotide sequences encoding a plurality of antibody polypeptides or antibody polypeptide fragments. For example, the library or vectors or expression cassettes may comprise a plurality of nucleic acid sequences comprising nucleotide sequences encoding a plurality of scFv antibody polypeptide fragments. Such a plurality of transformed yeast cells may be used to screen for scFv antibody polypeptide fragments that bind one or more particular antigens.

In certain embodiments, a first plurality of haploid yeast cells (e.g., a Yarrowia cell such as Yarrowia lipolytica) is transformed with a library of vectors or expression cassettes, which library comprises a plurality of nucleic acid sequences comprising nucleotide sequences encoding a plurality of antibody polypeptides or antibody polypeptide fragments, and a second plurality of haploid yeast cells (e.g., a Yarrowia cell such as Yarrowia lipolytica) is transformed with a library of vectors or expression cassettes, which library comprises a plurality of nucleic acid sequences comprising nucleotide sequences encoding a plurality of antibody polypeptides or antibody polypeptide fragments. In certain embodiments, the first and second pluralities of haploid yeast cells are transformed with the same library. For example, first and second pluralities of haploid yeast cells may be transformed with a library comprising nucleotide sequences encoding both heavy and light chain antibody polypeptides or fragments. In certain embodiments, the first and second pluralities of haploid yeast cells are transformed with a different library. For example, the first plurality of haploid yeast cells may be transformed with a library comprising nucleotide sequences encoding heavy chain antibody polypeptides or fragments, while the second plurality of haploid yeast cells may be transformed with a library comprising nucleotide sequences encoding light chain antibody polypeptides or fragments.

In certain embodiments, a first and second plurality of haploid yeast cells transformed with a library are mated to each other to form a plurality of diploid yeast that comprise vectors or expression cassettes from each library. For example, the first plurality of haploid yeast cells transformed with a library comprising nucleotide sequences encoding heavy chain antibody polypeptides or antibody polypeptide fragments may be mated to a second plurality of haploid yeast cells transformed with a library comprising nucleotide sequences encoding light chain antibody polypeptides or antibody polypeptide fragments to generate a plurality of diploid yeast cells comprising both heavy and light chain antibody polypeptides or antibody polypeptide fragments. Such a plurality of diploid yeast cells may be used to screen for antibody polypeptides or fragments that binds one or more particular antigens. Such embodiments are advantageous in that they permit screening of a large variety of different combinations of heavy and light chain antibody polypeptides or antibody polypeptide fragments.

In certain embodiments, the binding specificity of an antibody polypeptide or antibody polypeptide fragment for a particular antigen is improved or optimized. Directed evolution or affinity maturation can be used to improve or optimize the binding specificity of an antibody polypeptide or antibody polypeptide fragment. For example, Fujii (Antibody Engineering, Vol. 248, pp. 345-359, 2004, incorporated herein by reference in its entirety) describes the process of affinity maturation for antibodies. Similarly, Boder et al. (Proc. Natl. Acad. Sci. U.S.A. September 26; 97(20):10701-5, 2000, incorporated herein by reference) describes directed evolution of scFv fragments. These and other techniques can be employed in improving or optimizing the binding specificity of an antibody polypeptide or antibody polypeptide fragment

In certain embodiments, a nucleic acid sequence comprising a nucleotide sequence encoding an antibody polypeptide or fragment that binds, or is suspected of binding, a particular antigen may be isolated. Such a nucleic acid sequence may then be modified by changing one or more nucleotide residues. In certain embodiments, the nucleic acid sequence is part of a vector or expression cassette. The modified nucleic acid or acids may then be tested for the ability to bind an antigen (e.g., the original antigen or another different antigen). For example, modified nucleic acids may be introduced (e.g., by transformation) into a yeast cell, which yeast cell is incubated under growth conditions (e.g., Yarrowia operating conditions) such that an antibody polypeptide or antibody polypeptide fragment thereof is expressed on its cell surface. The yeast may then be contacted with an antigen of interest and binding may be tested.

A variety of techniques for modifying nucleic acid sequences are known in the art, any of which can be used in accordance with the presently disclosed methods and compositions. For example, radiation, chemical mutagens, error-prone PCR or saturation mutagenesis may be used. Other techniques can be found in Sambrook, J., Fritsch, E. F., and Maniatis, T. Molecular Cloning: A Laboratory Manual. 2.sup.nd ed., Cold Spring Harbor Laboratory, Cold Spring Harbor Laboratory Press, Cold Spring Harbor, N.Y., 1989, the contents of which are incorporated herein by reference in their entirety. Those of ordinary skill in the art will be aware of suitable techniques for modifying nucleic acid sequences.

A variety of techniques for testing binding of a cell displaying an antibody polypeptide or antibody polypeptide fragment to a given antigen are known in the art, any of which can be used in accordance with the presently disclosed methods and compositions. As one non-limiting example, an ELISA assay may be used.

In certain embodiments, a Yarrowia cell comprises a parent vector or parent expression cassette encoding an antibody polypeptide or antibody polypeptide fragment, which antibody polypeptide or antibody polypeptide fragment is displayed on the cell surface and binds a particular antigen (e.g., a target polypeptide). In certain embodiments, the parent vector or parent expression cassette is isolated and subjected to modification as described above to generate a one or more modified vectors or expression constructs. In certain embodiments, such a modification occurs in a nucleotide sequence encoding the antibody polypeptide or antibody polypeptide fragment. The one or more modified vectors or expression constructs may then be transformed into one or more second Yarrowia cells that lack the parent vector or parent expression cassette. For example, the one or more modified vectors or expression constructs may then be transformed into a plurality of Yarrowia cells that are grown under Yarrowia cell operating conditions to generate a Yarrowia antibody polypeptide yeast library, the members of which display a plurality of modified antibody polypeptides or antibody polypeptide fragments thereof on their surfaces. Members of the Yarrowia antibody polypeptide yeast library may then be tested for their ability to bind a particular antigen, e.g. the antigen that was bound by the antibody polypeptide or antibody polypeptide fragment encoded by the parent vector or parent expression cassette. Modified vectors or expression cassettes from those members of the Yarrowia antibody polypeptide yeast library that exhibit improved binding to the antigen (e.g., exhibit greater or more specific affinity or avidity) may be isolated. In certain embodiments, this sequence of steps is repeated one or more times. In certain embodiments, this sequence of steps is repeated until an antibody polypeptide or antibody polypeptide fragment that exhibits a desired level of binding is obtained.

In certain embodiments, an antibody polypeptide or antibody polypeptide fragment encoded by a nucleotide sequence in a parent vector or parent expression cassette is modified such that the modified antibody polypeptide or fragment exhibits improved or optimized binding to the same antigen bound by the antibody polypeptide or antibody polypeptide fragment encoded by the parent vector or parent expression cassette. In certain embodiments, an antibody polypeptide or antibody polypeptide fragment encoded by a nucleotide sequence in a parent vector or parent expression cassette is modified such that the modified antibody polypeptide or fragment exhibits improved or optimized binding to a different antigen bound by the antibody polypeptide or antibody polypeptide fragment encoded by the parent vector or parent expression cassette. For example, an antibody polypeptide or fragment known to bind a first antigen may be modified such that its antigen specificity is altered.

Those of ordinary skill in the art will be aware of other applications, and will be able to employ compositions and methods disclosed herein for use in such applications.

### Kits

In certain embodiments, kits comprising one or more compositions described herein are provided. In certain embodiments, kits for performing one or more methods described herein are provided. In certain embodiments, a kit comprises components for expressing a polypeptide of interest, e.g., an antibody polypeptide or antibody polypeptide fragment, an anchor polypeptide, or both, on the surface of a yeast cell. For example, a kit may comprise one or more expression cassettes, vectors, yeasts, and/or components for transforming or culturing yeast. In certain embodiments, an expression cassette, a vector, a yeast, and/or a component for transforming or culturing yeast is one such as is described in the present specification.

In certain embodiments, a kit comprises an expression cassette or vector comprising a nucleic acid sequence comprising a nucleotide sequence encoding an antibody polypeptide or antibody polypeptide fragment, an anchor polypeptide, or both. In certain embodiments, a kit comprises a yeast such as a Yarrowia cell, e.g. a Yarrowia lipolytica cell. In certain embodiments, a Yarrowia cell of a kit is competent for transformation. In certain embodiments, a Yarrowia cell of a kit is packaged with one or more components that can be used to make the Yarrowia cell competent for transformation.

In certain embodiments, a kit comprises written instructions for use of an expression cassette, vector or other component of the kit, e.g., written instructions for using the expression cassette, vector or other component of the kit to express a polypeptide of interest (e.g., an antibody polypeptide or antibody polypeptide fragment, an anchor polypeptide, or both) on the surface of a yeast cell.

### EXAMPLES

### Example 1

### Materials and Methods

Strains Used:

E. coli MC 1061 was used for standard DNA amplification and cloning. Yarrowia lipolytica POld (MatA, leu2-270, xpr2-322), POld (MatA, ura3-302, leu2-270, xpr2-322) and POld (MatA, ura3-302, leu2-270, Ade2-844, xpr2-322) were used as recipients for vector transformation.

ScFv Expression Plasmids:

Four synthetic constructs were made to allow SfiI/NotI cloning of a scFv fragment upstream of a molecular anchor sequence. N-terminally of the SfiI restriction site, a BsmI restriction site was added for fusion at the C-terminus of either LIP2pre or LIP2prepro in the final expression plasmids. Downstream of the NotI restriction site, a c-Myc tag was added followed by a (Gly4Ser)₃ linker and NdeI & AvrII restriction sites to exchange anchorage domains. For anchorage, the following Yarrowia codon optimized sequences were inserted between the NheI and AvrII sites into the synthetic construct: 1) C-terminal end (960 bp) of S. cerevisiae SAG1 (ID 853460), 2) S. cerevisiae AGA2 (ID 852851) or 3) the C-terminal end (333 bp) of Yarrowia lipolytica CWPI (Accession Number AY084077). A second synthetic construct was made in which the AGA2 molecular anchor was situated N-terminally of the scFv. Here, codon optimized mature S. cerevisiae AGA2 was preceded by an BsmI site and followed by a (Gly4Ser)₃ linker, SfiI/NotI surrounded scFv coding sequence, c-myc and 6-his epitope tags and a AvrII restriction site. The complete synthetic constructs were digested with BsmI (T4) and AvrII cloned into SacII(T4)/AvrII-digested pYLPLXL2pre. For expression of AGA1, codon optimized mature S. cerevisiae AGA1 preceded by BsmI and followed by AvrII was digested with BsmI(T4) and AvrII and cloned into SacII(T4)/AvrII-digested pYLPUXL2pre.

To allow soluble expression of scFv fragments a Yarrowia, a codon optimized secretion construct was made synthetically. This construct contained the V5 and 6-his epitope tags preceded at the 5′ end by SfiI/NotI restriction sites for scFv cloning. This construct was digested with BsmI(T4) and AvrII and cloned into SacII(T4)/AvrII-digested pYLPUXL2pre. Codon optimized trastuzumab scFv and 4-4-20 scFv, as well as the anti-HEL scFv's D1.3 and M3, were synthesized and cloned between the SfiI and NotI restriction sites into the described plasmids. Anti-fluorescein 4-4-20 antibody has served as a model protein for the development of a S. cerevisiae surface display platform (Boder, E. T. & Wittrup, K. D., Nat. Biotechnol. 15, 553-7, 1997, incorporated herein by reference in its entirety). Trastuzumab (Herceptin®), which binds to the cell surface antigen HER-2/neu proto-oncogene is clinically approved for the treatment of breast cancer (Cho et al., Nature, 421, 756-760, 2003, incorporated herein by reference in its entirety).

Fab Expression Constructs:

For the heavy chain expression plasmids, the Yarrowia codon optimized heavy chain constant region CH1 domain was cloned using SfiI and NotI into the four synthetic constructs as described for scFv cloning. cDNA for VH was then cloned using SfiI and NheI into these plasmids. Finally, Fab expression cassettes were cloned into pYLPLXL2pre similarly to what was done for scFv.

The light chain expression plasmid was built on the scFv expression plasmid. Therefore Yarrowia codon optimized Cx1 (light chain constant region kappa) was inserted with SfiI and NotI into this vector. cDNA for the VL was then cloned using SfiI and BsiWI into this plasmid.

Trastuzumab and 4-4-20 variable domains were amplified by PCR from scFv expression plasmids with the addition of the required restriction sites for cloning into the developed Fab expression plasmids. The final plasmids were transformed into suitable Yarrowia lipolytica strains as described above to create a fully complemented final strain.

Growth Conditions:

Yarrowia lipolytica strains were cultivated either on rich YPD medium (1% yeast extract, 1% bactopepton, 1% glucose) or on minimal medium supplemented with CSM (MSM; 0.67% yeast nitrogen base without amino acids and ammonium sulphate, 0.4% NH4Cl, 0.079% CSM) and supplemented with glucose 2% or oleic acid 2% as carbon source, in 50 mM phosphate buffer, pH 6.8, at 28° C. For experiments on pH testing, 50 mM phosphate-citrate buffer was used at pH 5 or pH 3.

To induce cell surface display, yeast cells were grown for 24 hours in minimal glucose medium at 28° C. and at 180 rpm. The following day, the OD600 of the culture was measured; cells were washed twice with dH₂O, resuspended at an OD600 of 0.1 in minimal oleic acid medium and grown for 16 hours at 20° C. and at 180 rpm. Cell were grown either as 5 mL cultures in 50 mL FALCON tubes or as 20 mL cultures in 250 mL baffled shake flasks.

Flow Cytometry:

Surface expression was demonstrated by indirect immunostaining with an antibody against the c-Myc or V5 epitope. Therefore, after induction, 2×10⁶ cells in 1 ml PBS (pH7.2) supplemented with 0.1% BSA (PBS/BSA) were incubated for 30 min with 1 μg/ml anti c-Myc antibody (Sigma) or anti-V5 antibody (Invitrogen). If appropriate, biotinylated HEL (Sigma) or recombinant HER2-Fc chimeric protein (R&D Systems) were used. EZ-Link Micro Biotinylation Kits from Pierce were used for the biotinylation of HEL. Then cells were washed with ice-cold PBS/BSA, and incubated for 30 minutes with secondary detection reagents. Goat anti-mouse Alexa-488 or phycoerythrin conjugated antibodies were used to detect the bound anti-c-Myc or anti-V5 antibody. For the detection of biotinylated antigen, detection was with streptavidin-phycoerythrin. Cells were washed twice with ice-cold PBS/BSA prior to analysis on a FACSCalibur flow cytometer.

Kd Determination:

Cells were grown and induced as described before. Aliquots of 1×10⁶ cells in 200u1 PBS/BSA were incubated with the appropriate antigen at a range of concentrations from 0.01 nM to 104, and were allowed to approach equilibrium at 25° C. by incubation for 60 min. Cells were next pelleted by centrifugation, washed in ice-cold PBS/BSA, and resuspended in 1 ml ice-cold PBS/BSA for analysis on a FACSCalibur flow cytometer. The mean fluorescence intensity of the cells was recorded. A nonlinear least-squares curve fit was used to determine the equilibrium dissociation constant (Kd) from the fluorescence data.

Construction of Diversified Repertoires Using Error Prone PCR:

The anti-HEL scFv fragment D1.3 was randomly mutated using error-prone PCR as described previously (see Chao et al., Nat. Protoc. 1, 755-68, 2006, incorporated herein by reference in its entirety). Briefly, the scFv ORF was amplified from pYLPUXL2preA2D1.3 using primers pPOX2Fw and zetaRv (Chao et al., Nat. Protoc. 1, 755-68, 2006). After purification, the PCR products were digested with SfiI and NotI. The digested products were gel-purified and cloned into similarly treated (digested with SfiI and NotI) vector containing wild-type D1.3. Plasmid DNA was prepared from these libraries using a Qiagen plasmid purification kit and was subsequently transformed into the Yarrowia strain pO1d as described above.

Library Selection:

The mutant D1.3 repertoire was grown and antibody expression was induced for 16 h as described above. The repertoire was labeled with anti-c-Myc (1 μg/ml) and 300 nM biotinylated HEL until equilibrium was reached (3 h), followed by a competition with unlabeled HEL for 20 minutes. Next cells were labeled with a secondary Alexa-488 labeled goat anti-mouse IgG (1 μg/ml) and streptavidin-phycoerythrin (1 μg/ml). Cells were washed twice with 1 ml PBS/BSA following all incubation steps. After the final wash, cells were kept on ice to prevent antigen dissociation. Samples were sorted on an Epics Altra flow cytometer with a sorting rate of approximately 2000 cells/s. Cells were sorted in three consecutive rounds with increasing stringency by gating a smaller percentage of the highest antigen binding population. Sequence analysis of the selected clones revealed that two clones (clones 13 and clone 38) contained mutations [I160V] (clone 13) and [I160V; T228A] (clone 38). These clones were assessed for antigen binding by equilibrium titration and showed 1.8 and 2.4 fold improved affinity, which lies in the same range as for the M3 mutant (see FIG. 18).

### Example 2

### Construction of a Set of scFv, Fab and Full Length IgG Display Plasmids

A generic surface display platform was created, to allow display of scFv fragments using different anchoring molecules. A total of four display plasmids was created allowing display of a scFv fragment as an 1) N-terminal fusion to the C-terminal part of S. cerevisiae Sag1p (320 C-terminal AA; A1), 2) an N-terminal fusion to S. cerevisiae Aga2p (A2), 3) an N-terminal fusion to the C-terminal part of Yarrowia lipolytica CwpIp (110 C-terminal AA; A3) and 4) a C-terminal fusion to Aga2p (A4). Expression was driven by the inducible pPOX2 promoter, and the LIP2pre leader sequence was appended N-terminally to the scFv to drive processing of each polypeptide through the secretion apparatus ultimately leading to a properly processed surface displayed protein. As a variant, the LIP2 prepro was also used as a leader for the trastuzumab scFv in alpha-aggltinin fusion (A1) to allow comparison of display levels. This experimental strategy was based on the reasoning that the use of multiple display formats would not only increase the chances of success, but would also allow the display of antibody fragments with either free carboxy or amino termini, depending on whether the anchor was fused to the N- or C-terminus of the scFv or Fab fragment, a feature that was previously shown to affect binding characteristics of a displayed scFv (Wang, Z. et al., Protein Eng. Des. Sel. 18, 337-43, 2005, incorporated herein by reference in its entirety). The addition of an epitope tag (c-Myc) allowed monitoring of display of each polypeptide and permitted normalized selection.

For Fab display, the heavy chain Fab fragment was anchored to the yeast surface using the same anchoring molecules as described for scFv fragments (A1-4), whereas the light chain Fab fragment was expressed as a soluble fragment (FabLC). To allow both chains to be present in stoichiometric amounts, both expression cassettes were driven by the inducible pPOX2 promoter, using LIP2pre as a leader sequence. The presence of different epitope tags for Fab heavy chain (CH1-VH), (c-Myc) and light chain, (C-terminal V5 and 6-his epitope tags) allowed for simultaneous and independent visualization of each polypeptide.

For full length IgG display, the full length trastuzumab heavy chain was anchored to the yeast cell surface using two of the anchoring molecules as described for scFv and Fab: A2 and A4 (N- and C-terminal tethering of AGA2 respectively). To allow both chains to be present in stoichiometric amounts, both expression cassettes were driven by the inducible pPOX2 promoter, using LIP2pre as a leader sequence. The presence of different epitope tags for heavy chain (HC), (c-Myc) and light chain (LC), (C-terminal V5 and 6-his epitope tags) allowed for simultaneous and independent visualization of each polypeptide.

All display cassettes were codon optimized for Yarrowia lipolytica since it was shown that codon optimization generally results in a twofold improvement of heterologous protein expression level. The display systems were analyzed using a panel of two well-characterized antibodies: anti-fluorescein 4-4-20 antibody, which has served as a model protein for the development of a S. cerevisiae surface display platform (Boder, E. T. & Wittrup, K. D., Nat. Biotechnol. 15, 553-7, 1997, incorporated herein by reference in its entirety) and Trastuzumab (Herceptin®), which binds to the cell surface antigen HER-2/neu proto-oncogene and is clinically approved for the treatment of breast cancer.

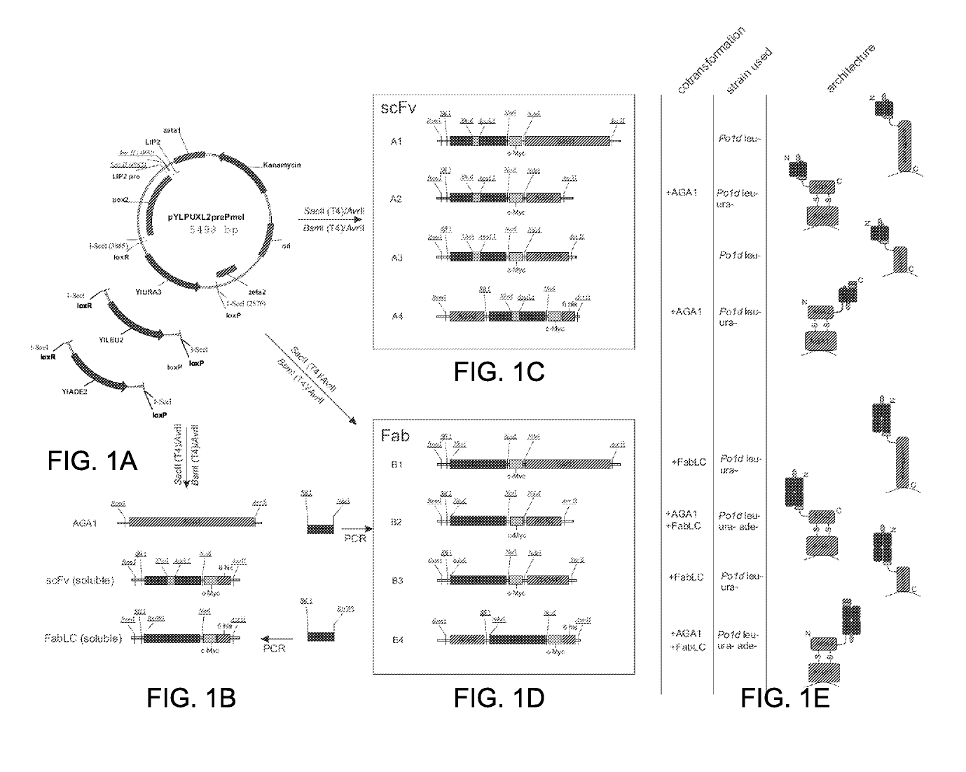

All vectors carried zeta elements (Long Terminal Repeats (LTRs) from the Ylt1 retrotransposon), which allowed the vectors to integrate either by homologous recombination in Y. lipolytica strains carrying Ylt1, or by nonhomologous recombination in strains devoid of this retrotransposon. All scFv expression constructs, as well as the Fab heavy chain (CH1-VH) expression constructs, carried the LEU2 auxotrophic marker. The Fab light chain fragment expression plasmids carried the URA3d1 marker. For display of the Aga2p fusion, an additional expression construct expressing the S. cerevisiae AGA1 was present. AGA1 is a heterodimerisation partner of AGA2. Therefore, two constructs were made (with auxotrophic markers URA3 and ADE2) to allow expression of AGA1 under pPOX2 promoter and using LIP2pre as a leader for the mature Aga1p. Transformation of the expression constructs resulted in every case in a fully complemented strain. FIG. 1 shows a schematic for the expression plasmids constructed for the display of scFv and Fab fragments.

### Example 3

### Improvement of Cellular Display

For initial experiments, positive transformants of display strains were grown overnight at 28° C. in 50 ml of YPD medium in 250 ml flasks. Thereafter, cells were washed in dH₂O, resuspended in oleic acid rich medium and grown for 48 hours at 28° C. in 250 ml flasks. In the initial experiments, no surface expression could be detected using immunological staining on the c-Myc epitope-tag and FACS analysis (data not shown). Therefore, different growth conditions were tested.

Many important cellular processes, including stress response and protein folding are affected by changing the growth temperature. Folding stress is generally decreased at lower cultivation temperatures, enabling more efficient heterologous protein secretion/surface display levels. See e.g., Dragosits, M. et al., J. Proteome Res., 2009, incorporated herein by reference in its entirety. Therefore surface expression levels of the scFv and Fab fragments were compared at induction temperatures of 20° C. and 28° C.

The cell wall is a highly adaptable organelle containing a highly diverse protein population. It has been shown in S. cerevisiae that the insertion of new macromolecules (e.g. GPI-anchored proteins) into the existing polymer network occurs mainly at the site of active cell wall biogenesis, i.e. at the site of the growing daughter cell (Klis, F. M., et al., Yeast 23, 185-202, 2006, incorporated herein by reference in its entirety). The molecular organization of the cell wall of Yarrowia lipolytica is believed to be similar to that of S. cerevisiae. It was tested whether growth at 20° C. would slow down cell wall formation, thus allowing more of the heterologous protein to accumulate at the site of cell wall biogenesis. Also, to study the effect of aeration, cells were grown in non-aerated 50 ml FALCON tubes, as well as 250 ml shake flasks. Finally, growth in minimal supplemented medium (MM) was tested. A strain displaying 4-4-20 alpha-agglutinin (Sag1p) was used for this experiment. As a control strain, a full size monoclonal trastuzumab antibody production strain was chosen (strain 1T2, containing no surface expression cassette).

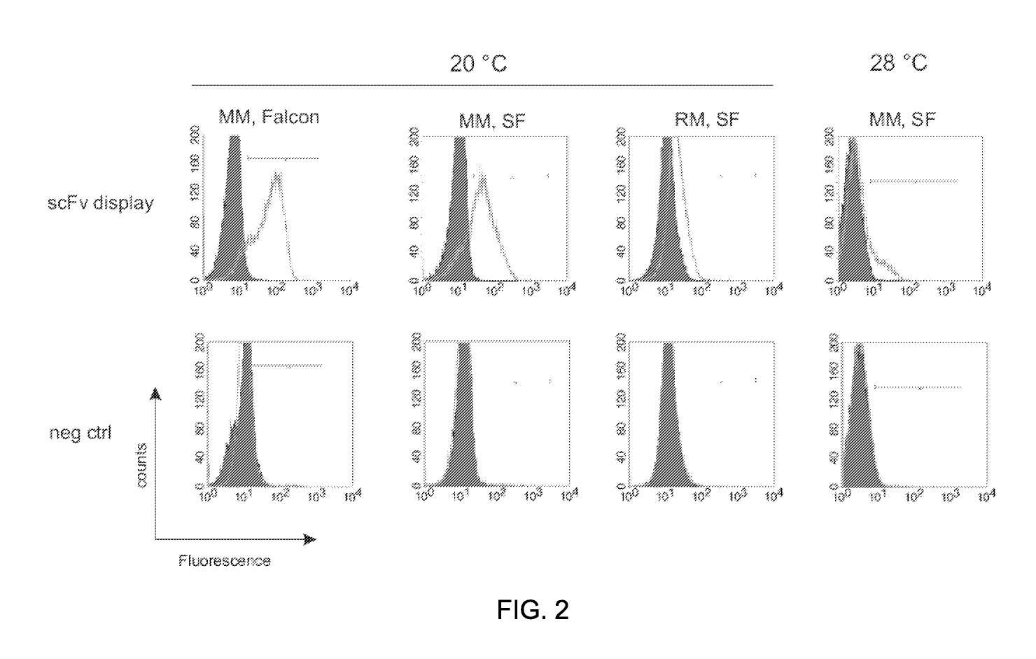

As depicted in FIG. 2 a large c-Myc positive population appeared upon FACS analysis when cells were induced for 20 hours at 20° C. in minimal supplemented medium both for FALCON (76%) and shake flask cultures (86%), with shake flask cultures showing slightly higher display levels (MFI (mean fluorescence intensity) differed by 2-fold). When cells were grown in MM at 28° C., only a small fraction of cells displayed the antibody fragments. Also, when cells were grown in RM at 20° C., no surface display was apparent. Upon analysis at 40 hours of induction, all c-Myc detection was abolished for all growth conditions tested (data not shown). Without wishing to be bound by theory, this could be explained by proteolysis of the displayed protein or hiding of the c-Myc epitope caused by morphological changes or changes in cell wall architecture.

### Example 4

### Effect of Induction Time on Surface Display Levels

Since c-Myc positive cells disappeared at longer induction times, a time-kinetics experiment was carried out to measure display levels at various induction times. Therefore FACS analysis of strain n1 (4-4-20 scFv Sag1 transformed pO1d) was carried out at 16, 20, 24, 32 and 43 hours induction.

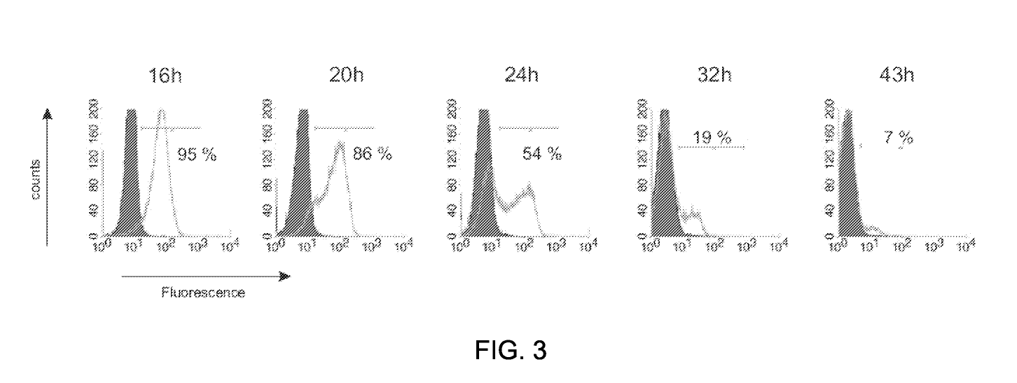

As depicted in the top panels of FIG. 3, a maximum expression level was reached at 16 hours of induction, with 95% of the cells showing moderate expression levels (10-fold above background). The relative proportion of cells expressing c-Myc decreased with longer induction times (95% after 16 hours, 86% after 20 hours, 53% after 24 hours, 19% after 32 hours, and 7% after 43 hours). Also, a decrease in the autofluorescence (5-fold) and in the mean fluorescence of the positive cells (20-fold) was observed during induction. Without wishing to be bound by theory, the decreased autofluorescence is likely the result of a decreased cell size. For the FSC/SSC (Forward-sideward scatter: These measurements are respectively indicative of cell size and granulosity of the cells), significant alterations were observed, which reflected drastic changes in the morphological development. Without wishing to be bound by theory, one explanation for these changes is that the cells undergo a yeast-hyphae transition during induction. As verified by microscopy, the cells formed more elongated structures upon longer induction, supporting this hypothesis. Importantly, in hyphal form the cell wall protein content was previously shown to be decreased, which could also explain lowered surface display levels.

### Example 5

### Effect of pH on Surface Display Levels

Y. lipolytica grows as a mixture of yeast-like and short mycelial cells. One factor regulating the dimorphic transition is the pH of the growth media (Ruiz-Herrera, J. & Sentandreu, R., Arch. Microbiol. 178, 477-83, 2002, incorporated herein by reference in its entirety). It has been described that mycelium formation is maximal at pH near neutrality and decreases as pH is lowered to become almost null at pH 3 (Id.).

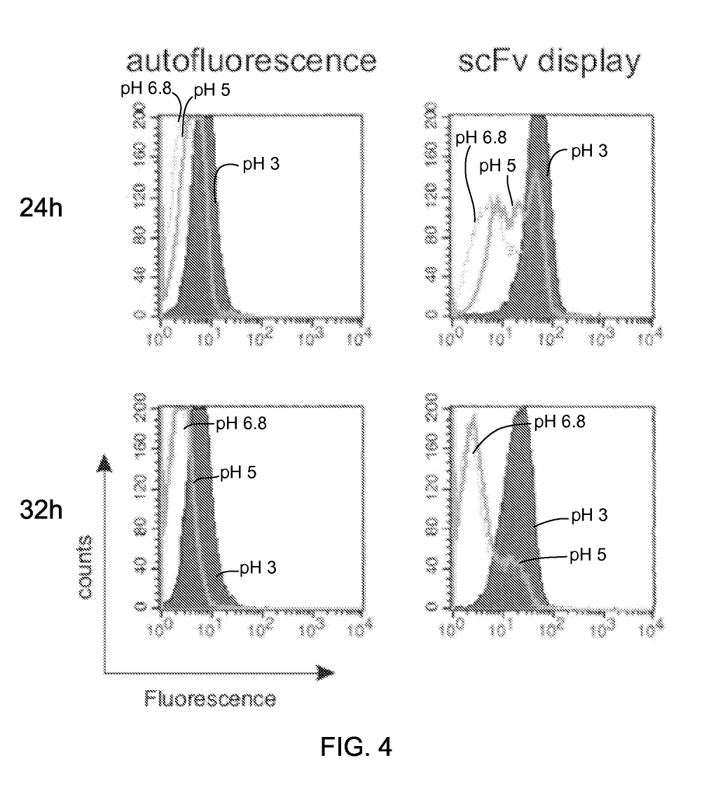

In an attempt to avoid yeast-hyphae transition during the initial phase of induction, a scFv display strain was grown at different pH values: pH 6.8, pH 5, and pH3. As depicted in FIG. 4, at 24 hours of induction a shift occurred for the cultures grown at pH 5 and 6.8 with 50% loss of displaying cells. On the contrary, at pH 3, 100% of cells retained cellular display. The overall display levels at pH 3 did not increase as compared to pH 6.8. At 32 hours induction a complete loss of c-Myc signal was observed at pH 5 and 6.8, while all cells retained scFv display at pH 3. Only a slight decrease in maximum expression levels was observed at pH 3 for longer induction times. Similar changes were seen for surface displayed Fab fragments (data not shown). Drastic differences were observed in FSC/SSC profiles between cultures grown at different pHs, reflecting morphological changes. At pH 3a yeast population that included very few mycelial cells was retained at longer induction times, whereas at pH 5 and 6.8 a more dispersed cell population was observed, probably reflecting a transition towards pseudo-hyphal growth.

In summary, this Example demonstrates that growth at low pH prolongs detection of surface display proteins but does not increase overall display levels.

### Example 6

### Expression Analysis of the Developed scFv, Fab and Full Length IgG Strains

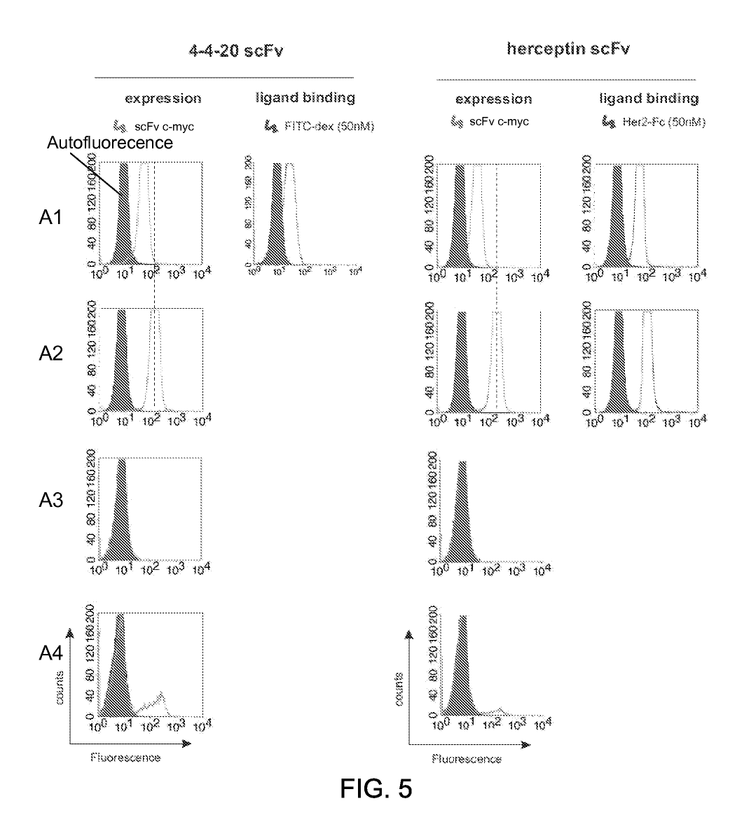

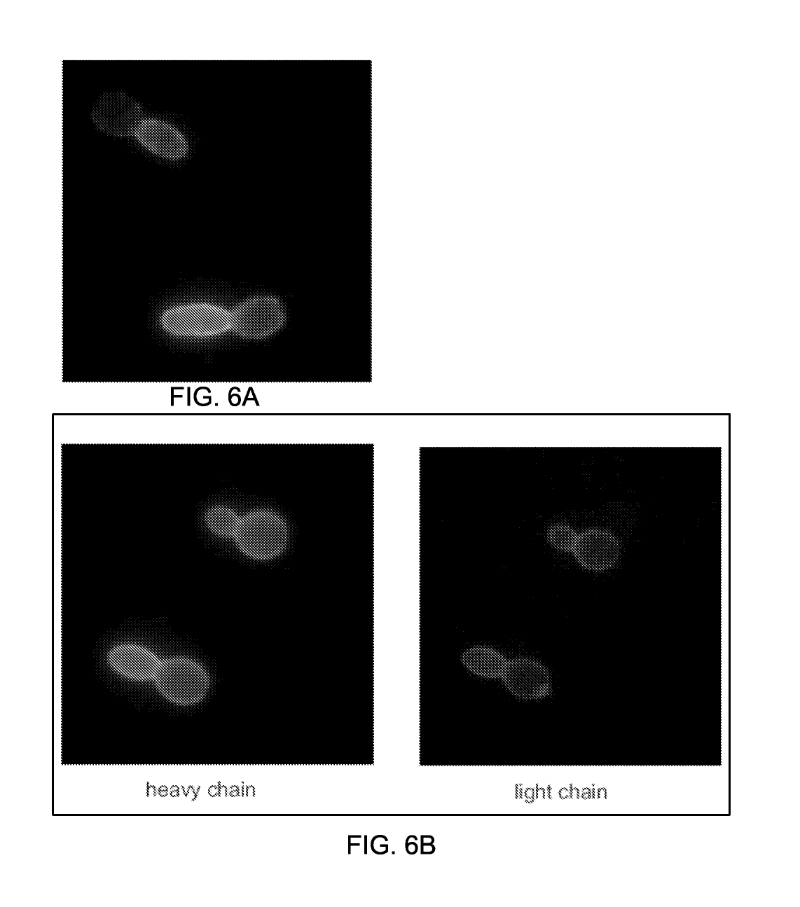

The new display system was validated with FACS using two different scFv fragment fusion proteins: 4-4-20 scFv and trastuzumab (Herceptin) scFv. scFv expression was verified by immunofluorescence microscopy and flow cytometric detection of the c-Myc tag, indicating expression and correct folding of the scFv product. FIG. 5 shows expression and ligand binding data for both scFv fragments in the different display formats. As shown, expression was seen for both scFv fragments for the N-terminal fusion to Sag1p (FIG. 5, histograms in row labeled “A1”) and Aga2p (FIG. 5, histograms in row labeled “A2”) with highest levels being achieved for Aga2p fusions (MFI was 30 fold above background). Importantly, in S. cerevisiae, there is always a negative population (40-80%) of cells present that do not express the surface protein, whereas this phenomenon was not observed when displaying scFvs in Yarrowia lipolytica using either fusion. Without wishing to be bound by theory, one potential explanation for this is that the expression cassette stably integrates into the genome of Y. lipolytica, in contrast to S. cerevisiae where episomal plasmids are used. For ligand-binding detection, biotinylated antigen was detected with streptavidin-phycoerythrin. The scFvs were also able to bind to antigen, confirming their correct processing and folding (see FIG. 5, columns labeled “ligand binding”). No expression could be detected for N-terminal fusion to CwpIp (FIG. 5, histograms in row labeled “A3”) and C-terminal fusion to Aga2p (FIG. 5, histograms in row labeled “A4”), even when multiple clones were tested. Several reasons could account for the absence of c-Myc detection in these cases. First, successful display of proteins in Yarrowia using CWPI has so far made use of hp4d promoter and Xpr2 pre as a leader sequence. One possibility is that differences in expression construct are responsible for the absence of expression that was observed. Second, it could be attributed to proteolysis of the epitope tags which could make the displayed protein undetectable. When the LIP2 prepro was used as a leader, an approximately 3 fold increase was seen for trastuzumab (Herceptin) Sag1p fusion (data not shown). Immunofluorescence microscopy clearly demonstrates the cell surface localization of the displayed scFv (see FIG. 6A).

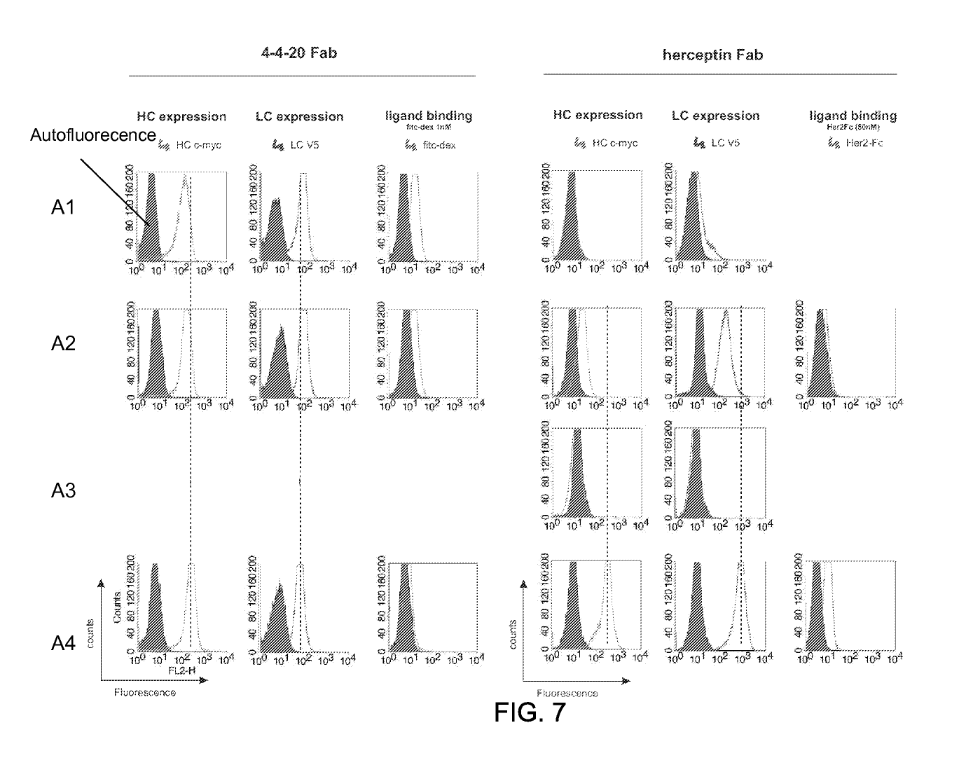

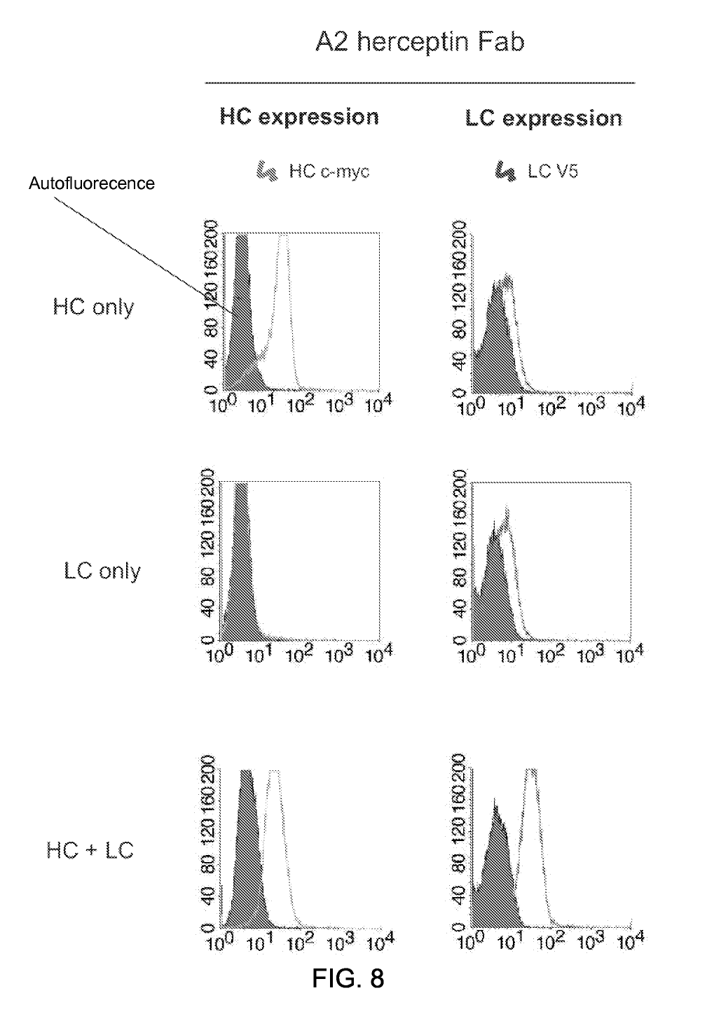

To evaluate if Yarrowia cells can functionally assemble heterodimeric Fab fragments on their surface, expression of two different Fab fragments (derived from the 4-4-20 and trastuzumab (Herceptin) antibodies) was induced followed by expression analysis by immunofluorescence microscopy and flow cytometry. Yarrowia strain pO1d was consecutively transformed with the expression cassettes for AGA1 (using ADE2 marker), heavy chain fragment (using URA3 marker) and light chain fragment (using LEU2 marker) to result finally in a fully complemented strain. Cells were grown and induced as described in Example 1. The Yarrowia cells were labeled for heavy chain and light chain expression by immunological staining against the fused epitope tags (c-myc for HC Fab fragment and V5 for LC-fragment) and antigen binding was assessed (see FIG. 7). For all constructs except fusion to CwpIp, display of both Fab heavy chain (CH1-VH) and light chain was confirmed. In all cases, 100% of the cell population expressed functional heterodimeric Fab fragments, confirming the results obtained with scFv fragments described above (see FIG. 7; a shift of the full peak, rather than the appearance of two peaks (one negative (autofluorescence) peak and one positive), was observed). Simultaneous labeling of HC and LC trastuzumab (Herceptin) Fab fragments using two color FACS analysis demonstrated the pairing of both chains on the surface of individual yeast cells (see FIG. 8, histograms in row labeled “HC+LC”). Moreover, in the absence of the Herceptin HC Fab fragment, the trastuzumab (Herceptin) LC fragment could not be detected on the surface of yeast cells, demonstrating the heterodimeric composition of the complex (see FIG. 8, histograms in middle row). Antigen binding was confirmed for both antibodies (FIG. 7, histograms in columns labeled “ligand binding”). However, the extent by which the antigen was bound differed according to the molecular organization of the antibody fusion. Also, when comparing the different display modes for the two antibody clones, changes in display efficiency were observed (FIG. 7, dotted lines). Immunofluorescence microscopy showed colocalization of both heavy and light chains (see FIG. 6B). In FIG. 6, Fab and scFv 4-4-20 antibody fragments were expressed. Detection was by c-myc staining for anchored heavy chain fragment and V5 staining for light chain fragment.

To evaluate if Yarrowia cells can functionally assemble a full length IgG on their surface, expression of a single IgG Herceptin (trastuzumab) was induced followed by expression analysis by immunofluorescence microscopy and flow cytometry. Therefore the expression cassettes of both chains were transformed to a single Yarrowia pO1d strain to generate a fully complemented strain, similarly as was done for Fab. The display was validated using FACS by staining heavy chain and light chain simultaneously (c-myc and V5 staining respectively).

FIG. 17 shows the flow cytometric analysis of full length trastuzumab (Herceptin) display in the two modes A2 and A4 (N- and C-terminal fusion to AGA2 respectively). As can be seen, all cells show expression of full length heavy chain and light chain simultaneously. A drastic improvement in display efficiency was observed for the case where the heavy chain is fused C-terminally of the AGA2 anchor as compared to N-terminal fusion, similarly as was observed for trastuzumab (Herceptin) Fab display.

### Example 7

### Engineering of Display Strains for Improved Expression of Antibody Fragments

The rate limiting steps in the production of antibody fragments (scFv and Fab) are often protein folding, disulfide bridge formation and functional assembly in the endoplasmic reticulum (ER). It has been shown that overexpression of molecular chaperones such as PDI and Kar2/Bip in S. cerevisiae had a positive effect on scFv production (Shusta, E. V., et al., Nat. Biotechnol. 16, 773-7, 1998, incorporated herein by reference in its entirety). Also, it has been shown in P. pastoris that PDI coexpression alleviates folding stress upon Fab overexpression, resulting in moderately increased production levels (Gasser, B., et al., Biotechnol. Bioeng. 94, 353-61, 2006, incorporated herein by reference in its entirety). However, in some cases chaperone coexpression resulted in no change or even a decrease in expression levels. Another possibility to improve antibody secretion is to induce the unfolded protein response (UPR) by overexpression of the HACI transcription factor; moderate improvements in Fab secretion have previously been reported (Id.).

It is known that cell surface display correlates well with secretory capacity as both surface displayed and secreted proteins migrate through the same secretory pathway (Shusta, E. V., et al., J. Mol. Biol. 292, 949-56, 1999, incorporated herein by reference in its entirety). As such, surface display levels function as an easy readout linking individual cells to expression levels.

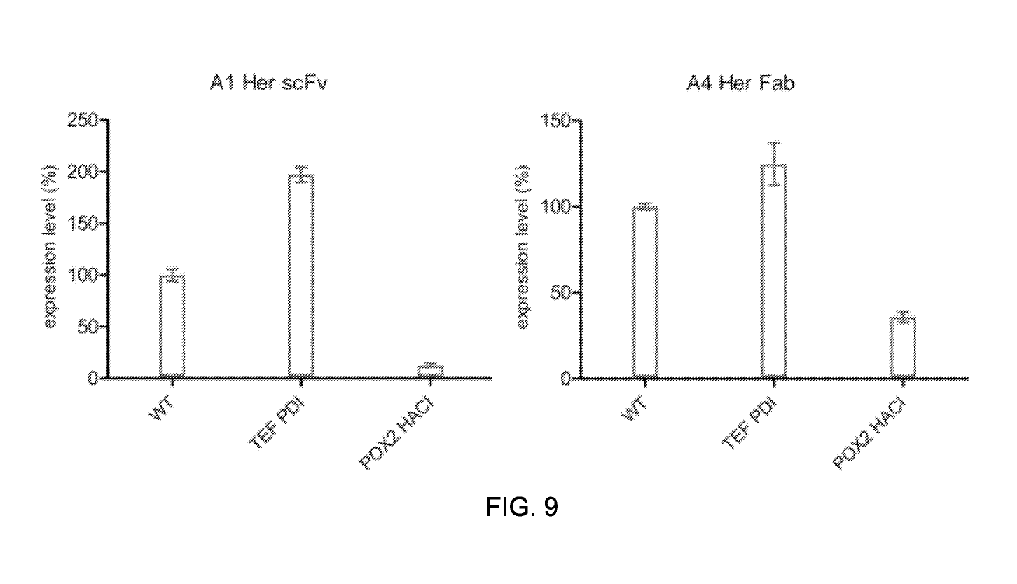

Here the effect of Yarrowia PDI and HACI expression on scFv and Fab production was tested for the first time in Yarrowia lipolytica using the display platform developed above. Yarrowia PDI was constitutively expressed under control of TEF promoter and Yarrowia HACI transcription factor was inducibly expressed under control of pPOX2 promoter. Both cassettes were cotransformed to trastuzumab (Herceptin) scFv and Fab displaying strains (described above), and correct genomic integration was confirmed by PCR. As shown in FIG. 9, constitutive PDI coexpression resulted in 2-fold increase, as measured by c-myc MFI, of trastuzumab (Herceptin) scFv-Sag1p display and a 1.2 fold increase of trastuzumab (Herceptin) Fab-Aga2 display. On the contrary, induced HACI coexpression resulted in a decrease of both scFv and Fab fragments. These results demonstrate that formation of disulfide bonds is a rate limiting step in the secretion of scFv and Fab fragments. However, induction of the UPR (unfolded protein response) pathway had a drastic negative impact. This was previously observed for display of a scFv (Rakestraw, A. & Wittrup, K. D., Biotechnol. Bioeng. 93, 896-905, 2006, incorporated herein by reference in its entirety) and could be explained by the fact that proteins that are not properly folded are sent to the ER degradation pathway (ERAD), which is also upregulated during UPR induction.

### Example 8

### Dose Response Curves for Displayed Trastuzumab (Herceptin) scFv

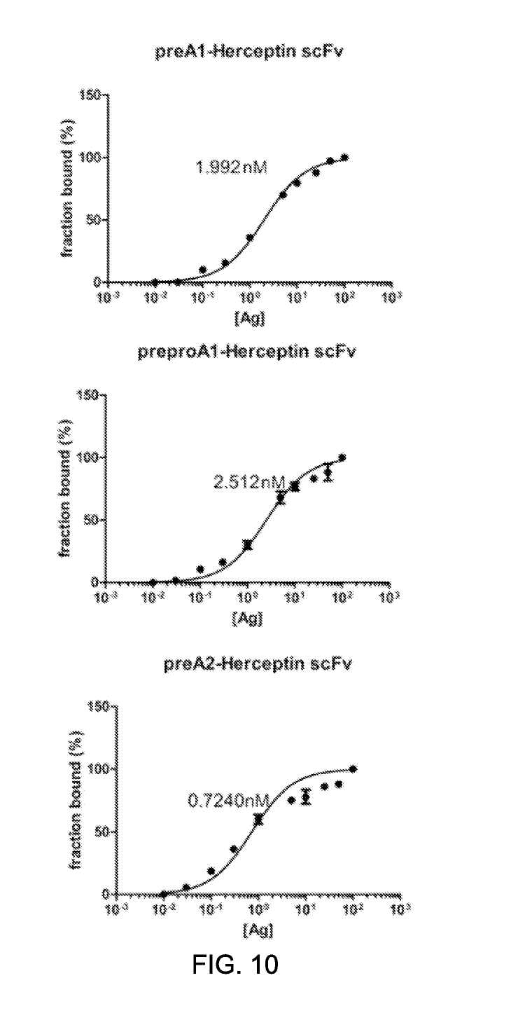

The binding affinity of trastuzumab (Herceptin) surface-displayed scFv fusion proteins was determined from equilibrium binding titration curves. Cells displaying either antibody fusion were incubated at 25° C. for 3 hours in varying concentrations of HER2-Fc chimeric protein. The mean fluorescence of the cell populations was measured by flow-cytometry. FIG. 10 shows the results of three independent titrations. The line graph labeled “preA1-Herceptin scFv” shows the dose response curve for trastuzumab (Herceptin) scFv fused as an N-terminal fusion to the to the C-terminal 320 amino acids of S. cerevisiae Sag1p and expressed with the Lip2pre leader sequence. The line graph labeled “preproA1-Herceptin scFv” shows the dose response curve for trastuzumab (Herceptin) scFv fused as an N-terminal fusion to the C-terminal 320 amino acids of S. cerevisiae Sag1p and expressed with the Lip2prepro leader sequence. The line graph labeled “preA2-Herceptin scFv” shows the dose response curve for trastuzumab (Herceptin) scFv fused to as an N-terminal fusion to S. cerevisiae Aga2p and expressed with the Lip2pre leader sequence. The Y axis shows fraction bound, which is calculated as MFI/(MFImax-MFImin), normalized, and expressed as a percentage. The equilibrium dissociation constant, Kd, was fit by nonlinear least squares. The affinity of yeast-displayed trastuzumab (Herceptin)-Sag1p fusion for HER2-Fc (Kd=1.9 nM) was 2.7 fold higher than that for trastuzumab (Herceptin)-Aga2p (Kd=0.7 nM).

### Example 9

### Validation of Yarrowia Display Platform as a Scaffold for Directed Evolution

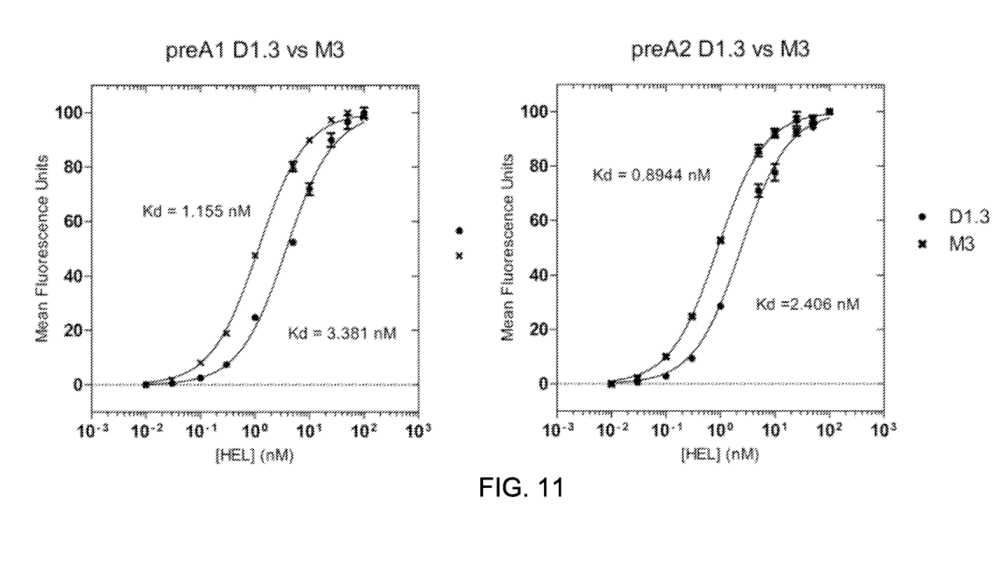

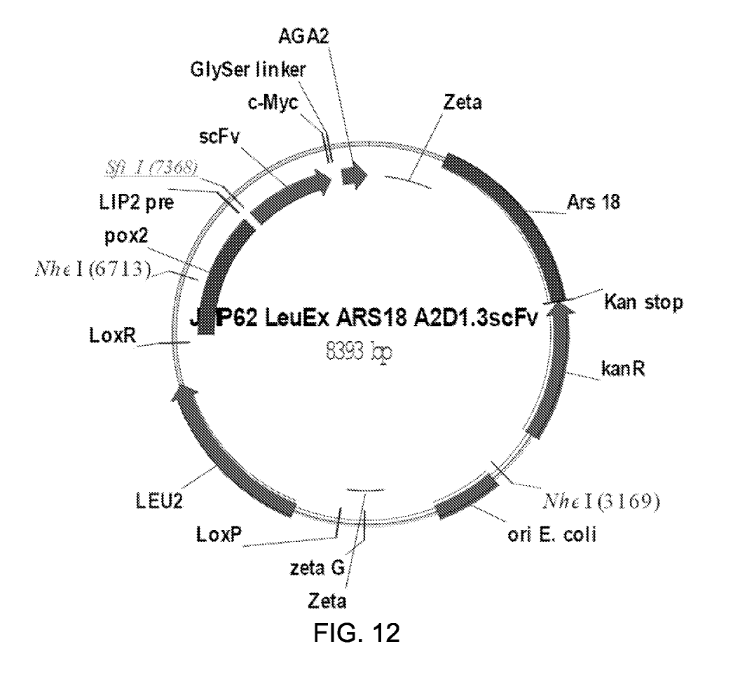

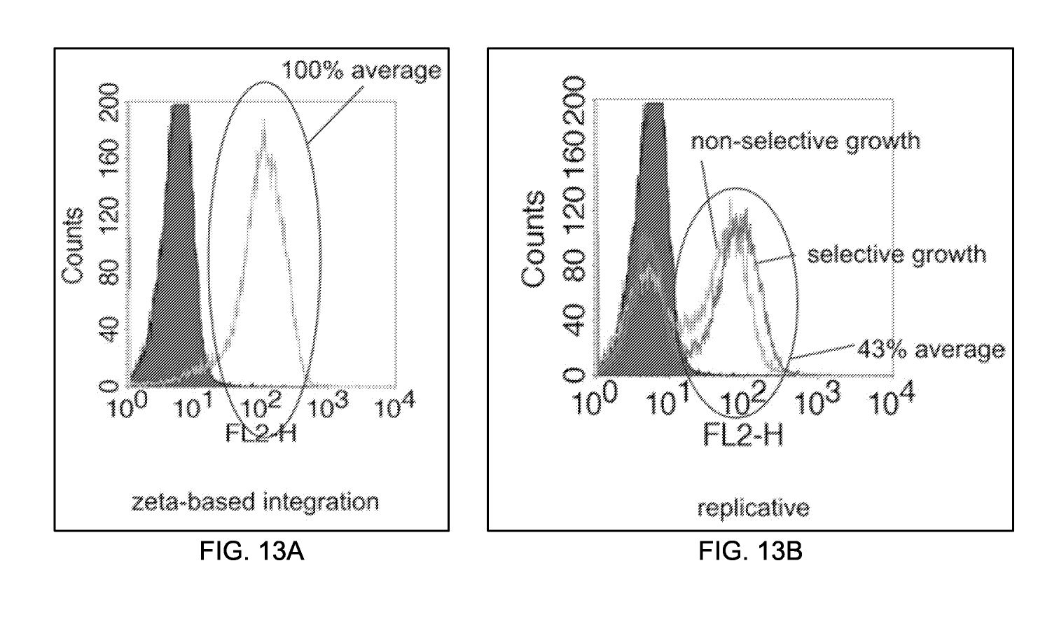

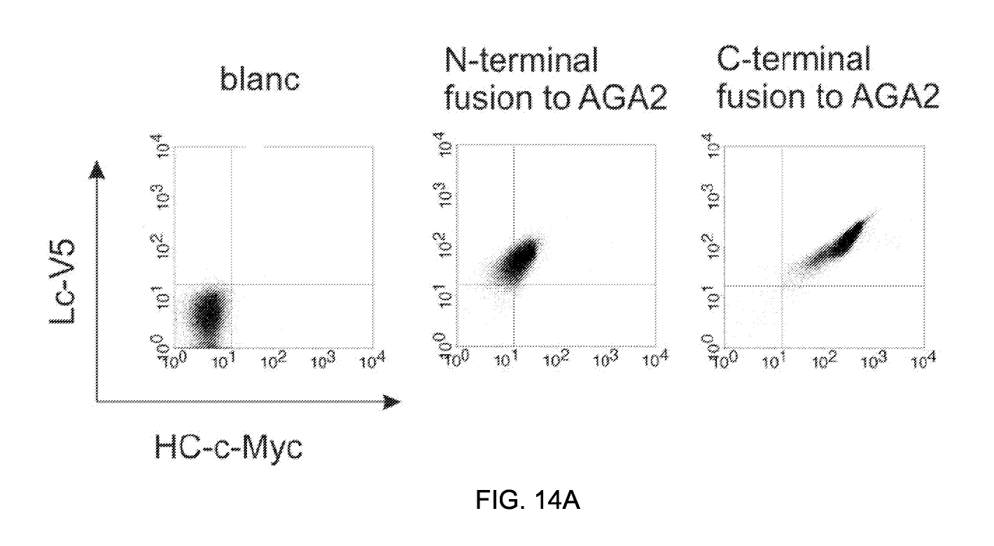

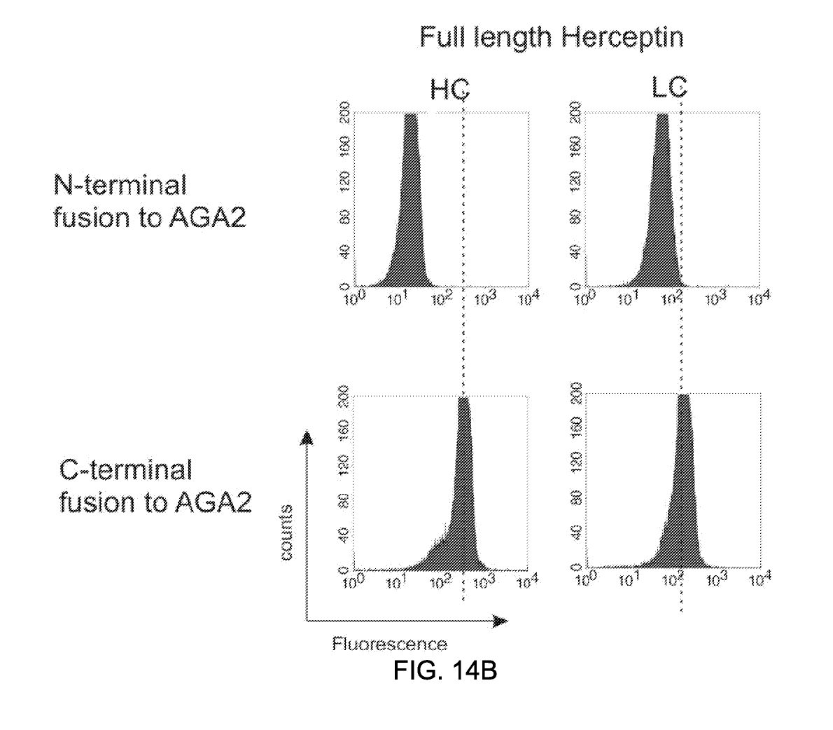

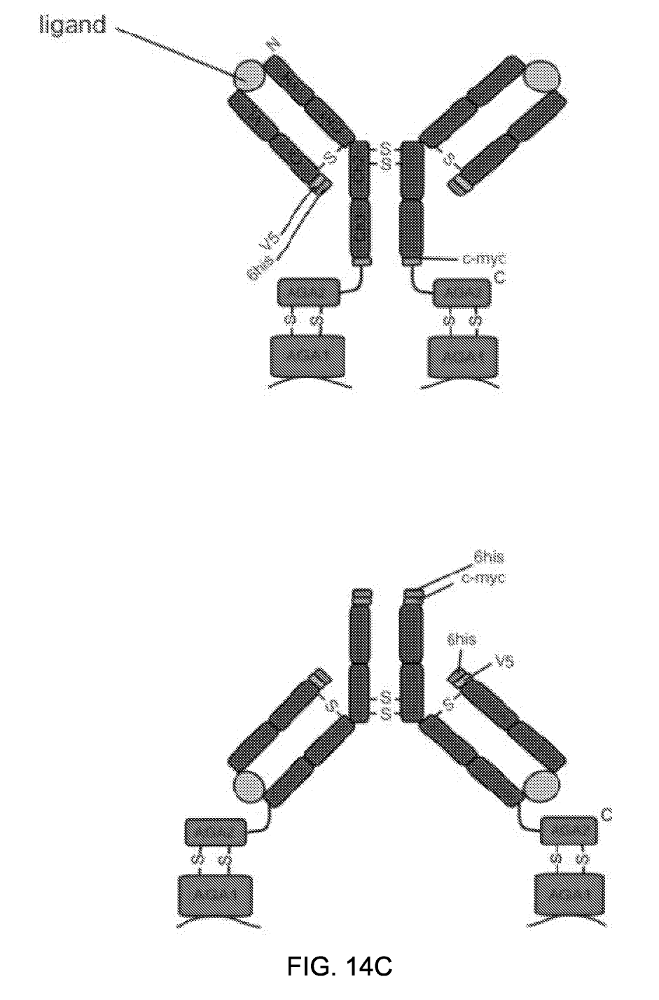

To obtain maximum directed evolution efficiency, a scaffold should be able to effectively discriminate between clones with only minor difference in affinity. Previously, it was shown that yeast display allows for fine discrimination between antibody clones with a 2-fold difference in affinity. See VanAntwerp, J. J. & Wittrup, K. D., Biotechnol. Prog. 16, 31-7, 2000, incorporated herein by reference in its entirety. Anti-hen egg lysozyme (HEL) scFv M3 has a 2-fold higher affinity for HEL than does anti-HEL scFv D1.3. The displayed polypeptides were expressed as Sag1p (line graph labeled “preA1D1.3 vs M3”) and Aga2p (line graph labeled “preA2 D1.3 vs M3”) fusion polypeptides. The D1.3 or M3 displaying cells were incubated with varying concentrations of biotinylated HEL. Next, the mean fluorescence was measured by flow cytometry. The binding affinity of each surface displayed antibody was determined by equilibrium binding titration curves. FIG. 11 shows the average results of three independent titrations, to which a curve was fit by nonlinear least squares. The affinity of D1.3 for HEL was determined to be 2.9 and 2.7 fold lower for Sag1p and Aga2p fusions respectively, as compared to the affinity of M3 for HEL.

This Example shows that the developed Yarrowia display scaffold effectively discriminates between clones with only minor differences in affinity, confirming the screening potential of this system.

### Example 10

### Model Enrichment Experiment Using FACS

Single pass enrichments using a mixture of yeast cells displaying the D1.3 and improved mutant M3 were performed. Cells displaying the M3 mutant scFv were additionally transformed with a hygromicin expression cassette. No significant effect on expression levels were observed. M3 cells were mixed in a ratio 1/1000 into background D1.3 cells and incubated until equilibrium was reached at an antigen concentration of 0.3 nM for optimal discrimination. Cells were sorted in high purity mode with a sorting window of approximately 0.1% (not shown). Enrichment factors were determined by titration on selective plates and a maximum enrichment of 800 was obtained. Enrichment can be calculated by replica plating on selective plates before and after enrichment.

### Example 11

### Surface Display Using Replicative Vector in Yarrowia lipolytica

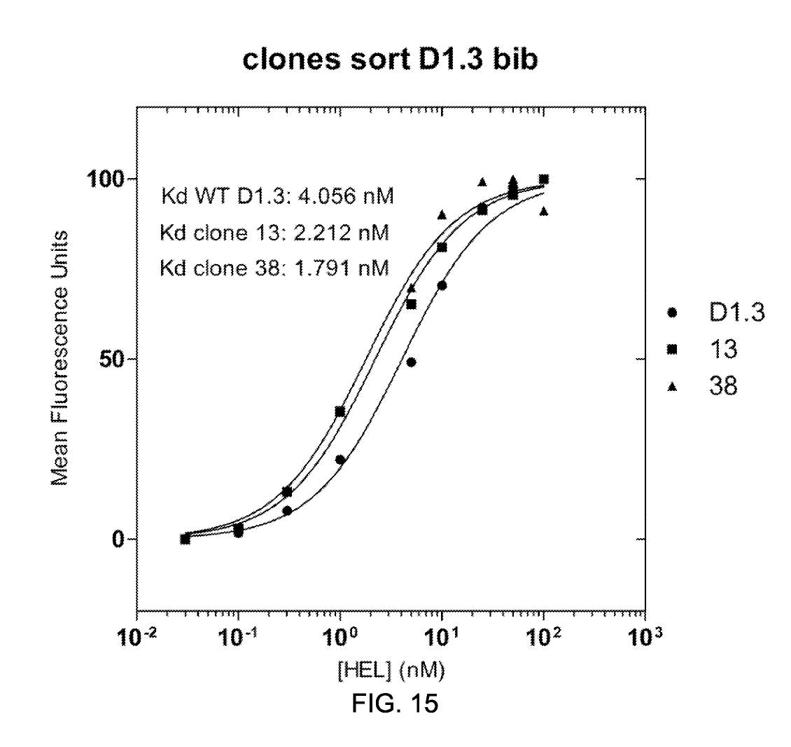

A replicative vector was constructed to contain a scFv-AGA2 expression cassette driven by a pPOX2 promoter and ARS18 for replicative propagation in Yarrowia lipolytica (FIG. 15). Upon transformation into a Yarrowia lipolytica strain containing the AGA1 expression cassette (for AGA1-AGA2 heterodimerisation), a transformation efficiency of 1.2×10⁶/μg was obtained. This efficiency was 20 higher as compared to what could be observed for random integration using zeta-based integration. For library construction high transformation efficiency is advantageous to obtain the desired complexity. To preserve plasmid propagation, cells were grown under selective conditions in the absence of leucine.

Expression studies were performed on ten clones grown under both selective (Minimal Medium supplemented with CSM-Leucine) and non-selective conditions (MM supplemented with CSM) using FACS. Contrary to what was observed for integrative plasmids (FIG. 16A), upon induction of cells transformed with a replicative vector, a population of cells exists that did not express scFv. This negative population existed even when the cells were grown under selective pressure (FIG. 16B), indicating that plasmid loss was not the basis for this observation. This phenomenon is similar as to what can be observed in S. cerevisiae using replicative plasmids for surface display. Analysis of the ten clones revealed that an average of 43% of the cells were positive for surface expression of the scFv (see FIG. 16B). The mean fluorescence intensity did not differ from the results obtained using integrative plasmids (i.e., the mean fluorescence average was in the same range).

### Example 12

### Enrichment Experiment Using FACS

An enrichment using a mixture of yeast cells displaying a diversified library of D1.3 was performed at an antigen concentration of 1 nM. Cells were sorted in high purity mode with a sorting window of approximately 0.1% (not shown). Three consecutive rounds of sorting were carried out. Two higher affinity clones were isolated: clone 1 showing an affinity of 1.7 nM (Ile160Val, Thr228Ala) and clone 2 showing an affinity of 2.2 nM (Ile160Val).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A Yarrowia cell comprising an expression cassette comprising:
a first promoter operably linked to a fusion sequence comprising a first nucleic acid sequence comprising a nucleotide sequence encoding a first antibody polypeptide, or a first antibody polypeptide fragment, fused in frame to an anchor polynucleotide sequence comprising a nucleotide sequence encoding an anchor polypeptide, wherein the first antibody polypeptide fragment comprises an antibody variable (V) region.

2. The cell of claim 1, wherein the first antibody polypeptide fragment is a scFv fragment.

3. The cell of claim 1, wherein the first antibody polypeptide or the first antibody polypeptide fragment is a Fab heavy chain or a Fab light chain.

4. The cell of claim 1, wherein the cell further comprises a second expression cassette comprising a second promoter operably linked to a second nucleic acid sequence comprising a nucleotide sequence encoding a second antibody polypeptide or a second antibody polypeptide fragment, wherein the second antibody polypeptide fragment comprises an antibody variable (V) region.

5. The cell of claim 4, wherein the first and second promoters are the same promoters.

6. The cell of claim 4 or 5, wherein the first antibody polypeptide or the first antibody polypeptide fragment is a heavy chain of an antibody Fab fragment and the second antibody poll/peptide or second antibody polypeptide fragment is a light chain of an antibody Fab fragment.

7. The cell of claim 4 or 5, wherein the first antibody polypeptide or the first antibody polypeptide fragment is a light chain of an antibody Fab fragment and the second antibody polypeptide or the second antibody polypeptide fragment is a heavy chain of an an antibody Fab fragment.

8. The cell of any of claims 1 or 1-7, wherein the anchor polynucleotide sequence is fused 3′ to the first nucleic acid sequence, such that a fusion polypeptide produced from the fusion sequence comprises an N-terminal antibody polypeptide or antibody polypeptide fragment and a C-terminal anchor polypeptide.

9. The cell of any of claims 1-7, wherein the anchor polynucleotide sequence is fused 5′ to the first antibody nucleic acid sequence, such that a fusion polypeptide produced from the fusion sequence comprises an N-terminal anchor polypeptide and a C-terminal antibody polypeptide or antibody polypeptide fragment.

10. The cell of any of claims 1-9, wherein the first promoter is constitutive.

11. The cell of any of claims 1-9, wherein the first promoter is inducible.

12. The cell of claim 11, wherein the first promoter is a POX2 or LIP2 promoter.

13. The cell of any of claims 1-9, wherein the first promoter is semi-constitutive.

14. The cell of claim 13, wherein the first promoter is an hp4d promoter.

15. The cell of claims 1-15, further comprising a leader nucleic acid sequence comprising a nucleotide sequence encoding a leader polypeptide, wherein the leader nucleic acid sequence is fused in frame with and 5′ to the anchor polynucleotide sequence and the first nucleic acid sequence.

16. The cell of claim 15, wherein the leader polypeptide is selected from the group consisting of; LIP2 pre, LIP2 prepro, XPR2 pre, and XPR2 prepro.

17. The cell of claims 1-16, further comprising a linker nucleic acid sequence comprising a nucleotide sequence encoding a linker polypeptide.

18. The cell of claim 17, wherein the linker nucleic acid sequence is fused in frame between the first anchor polynucleotide sequence and the first nucleic acid sequence.

19. The cell of claim 17, wherein the linker nucleic acid sequence is fused in frame between a nucleic acid sequence comprising a nucleotide sequence encoding a heavy chain variable region and a nucleic acid sequence comprising a nucleotide sequence encoding a light chain variable region.

20. The cell of claim 19, wherein the nucleic acid sequence comprising a nucleotide sequence encoding a heavy chain variable region comprises a nucleotide sequence encoding the heavy chain of a Fab fragment.

21. The cell of claim 19 or 20, wherein the nucleic acid sequence comprising a nucleotide sequence encoding a light chain variable region comprises a nucleotide sequence encoding a light chain.

22. The cell of any of claims 17-21, wherein the linker polypeptide comprises (Gly4Ser)₃ (SEQ ID NO:14) or (GlySer)₅ (SEQ ID NO:15).

23. The cell of any of claims 1-22, the expression cassette further comprising one or more additional nucleic acid sequences each comprising a nucleotide sequence encoding one or more epitope tags.

24. The cell of claim 23, wherein the one or more epitope tags are selected from the group consisting of: c-Myc, V5, hexahistidine, glutathione-S-transferase, streptavidin, biotin, hemagglutinin, Flag-tag, and E-tag.

25. The cell of any of claims 1-24, wherein the anchor polypeptide is selected from the group consisting of: an Aga1p polypeptide or fragment thereof, an Aga2p polypeptide or fragment thereof, and a Sag1p polypeptide or fragment thereof.

26. The cell of any of claims 1-24, wherein one or more coding sequences within one or more expression cassettes in the cell are codon optimized for expression in a Yarrowia cell.

27. The cell of any of claims 1-25, wherein one or more expression cassettes introduced into the cell are each in a vector.

28. The cell of claim 27, one or more of the vectors further comprising a zeta element.

29. The cell of claim 28, wherein the zeta element is a long terminal repeat of a retrotransposon.

30. The cell of claim 29, wherein the zeta element is a long terminal repeat of a Ylt1 or Tyl6 retrotransposon.

31. The cell of any of claims 27-30, wherein one or more of the vectors in the cell further comprise one or more autosomal replication elements.

32. The cell of claim 31, wherein at least one autosomal replication element comprises a centromere (CEN) and an origin of replication (ORI).

33. The cell of claim 32, wherein the centromere is CEN1 or CEN3 and the origin of replication is ORI1068 or ORI3018.

34. The cell of any of claims 1-33, wherein one or more of the vectors in the cell each further comprise and autonomously replicating sequence (ARS), wherein the ARS comprises a centromere and an origin of replication.

35. The cell of claim 34, wherein the ARS is ARS18.

36. The cell of claim 34, wherein the ARS is ARS68.

37. The cell of any one of claims 27-36, wherein one or more of the vectors in the cell further comprise one or more additional nucleic acid sequences, each additional nucleic acid sequence comprising a nucleotide sequence encoding one or more selectable markers.

38. The cell of claim 37, wherein the one or more selectable markers are selected from the group consisting of: LEU2 (leucine selectable marker, URA3d1 (uracil selectable marker), ADE2 (adenine selectable marker), Lys (lysine selectable marker), Arg (arginine selectable marker), Gut (glycerol utilization selectable marker), Trp (tryptothan selectable marker), G3p (glycerol-3-phosphate selectable marker), and hph (hygromycin B phosphotransferase selectable marker).

39. The cell of any one of claims 1-38, wherein the cell is a haploid cell.

40. The cell of any one of claims 1-38, wherein the cell is a diploid cell.

41. A method of expressing an antibody polypeptide or antibody polypeptide fragment in a Yarrowia cell, the method comprising:
induction incubating a first Yarrowia cell,
wherein the first Yarrowia cell comprises:
(a) a first vector comprising a first promoter operably linked to a fusion sequence comprising a first antibody nucleic acid sequence comprising a nucleotide sequence encoding a first antibody polypeptide, or a first antibody polypeptide fragment, fused in frame to an anchor polynucleotide sequence comprising a nucleotide sequence encoding an anchor polypeptide, wherein the first antibody polypeptide fragment comprises an antibody variable (V) region; or
(b) a first vector comprising a first promoter operably linked to a first antibody nucleic acid sequence comprising a nucleotide sequence encoding a first antibody polypeptide or a first antibody polypeptide fragment.

42. The method of claim 41, wherein the first Yarrowia cell comprises (a) and the nucleotide sequence of the first nucleic sequence encodes an antibody polypeptide fragment comprising a heavy chain variable region and a light chain variable region, wherein the first antibody polypeptide fragment comprises an antibody variable (V) region, and
wherein, after the induction incubation, the first antibody polypeptide fragment is expressed on the surface of the first Yarrowia cell.

43. The method of claim 41, wherein the first Yarrowia cell comprises (a) and has been converted to a second Yarrowia cell by the introduction into the first Yarrowia cell of a second vector comprising a second promoter operably linked to a second nucleic acid sequence comprising a nucleotide sequence encoding a second antibody polypeptide or a second antibody polypeptide fragment, wherein the second antibody polypeptide fragment comprises an antibody variable (V) region, and
wherein, after the induction incubation, a molecule comprising the first antibody polypeptide or first antibody polypeptide fragment and the second antibody polypeptide or the second antibody polypeptide fragment is expressed on the surface of the second Yarrowia cell.

44. The method of claim 42, wherein the first Yarrowia cell comprises (b) and has been converted to a second Yarrowia cell by the introduction into the first Yarrowia cell of a second vector comprising a second promoter operably linked to a fusion sequence comprising a second nucleic acid sequence comprising a nucleotide sequence encoding a second antibody polypeptide or a second antibody polypeptide fragment fused in frame to an anchor polynucleotide sequence comprising a nucleotide sequence encoding an anchor polypeptide, and
wherein, after the induction incubation, a molecule comprising the first antibody polypeptide or first antibody polypeptide fragment and the second antibody polypeptide or the second antibody polypeptide fragment is expressed on the surface of the second Yarrowia cell.

45. The method of claim 43 or 44, wherein the first promoter and the second promoter are identical promoters.

46. The method of claim 41, wherein the induction incubation is under two or more Yarrowia operating conditions.

47. The method of any of claim 41, 42, or 46, wherein the antibody polypeptide fragment is a scFv fragment.

48. The method of any of claims 43-46, wherein the first antibody polypeptide or the first antibody polypeptide fragment comprises an antibody heavy chain variable region or antibody light chain variable region and the molecule comprises an antibody heavy chain variable region and an antibody light chain variable region.

49. The method of claim 48, wherein
the first antibody polypeptide or the first antibody polypeptide fragment comprises:
a Fab heavy chain or a heavy chain V-CH1 fragment, or
an antibody light chain; and
the molecule comprises:
a Fab heavy chain or a heavy chain V-CH1 fragment, and
an antibody light chain.

50. The method of any of claims 41-44 and 46, wherein the second antibody polypeptide or the second antibody polypeptide fragment comprises an antibody heavy chain variable region or antibody light chain variable region and the molecule comprises an antibody heavy chain variable region and an antibody light chain variable region.

51. The method of claim 50, wherein
the second antibody polypeptide or the second antibody polypeptide fragment comprises;
a Fab heavy chain or a heavy chain V-CH1 fragment, or
an antibody light chain; and
the molecule comprises;
a Fab heavy chain or a heavy chain V-CH1 fragment, and
an antibody light chain.

52. The method of any of claims 43-46, 48, 49, 50 and 51, wherein the molecule, is a Fab fragment.

53. The method of any of claims 43-46, and 48-52, wherein the first Yarrowia cell is haploid, and wherein the introduction of the second vector into the first cell comprises mating the first haploid Yarrowia cell comprising the first vector with a donor haploid Yarrowia cell comprising the second vector,
wherein the first and the donor Yarrowia cells are of opposite mating types.

54. The method of any one of claims 41-53, wherein the nucleic acid sequence that comprises a nucleotide sequence encoding an antibody polypeptide or an antibody polypeptide fragment that is fused in frame to the anchor polynucleotide sequence is fused 5′ to the anchor polynucleotide sequence, such that a fusion polypeptide produced from the fusion sequence comprises an N-terminal antibody polypeptide or antibody polypeptide fragment thereof and a C-terminal anchor polypeptide.

55. The method of any one of claims 41-53, wherein the nucleic acid sequence that comprises a nucleotide sequence encoding an antibody polypeptide or an antibody polypeptide fragment that is fused in frame to the anchor polynucleotide sequence is fused 3′ to the anchor polynucleotide sequence, such that a fusion polypeptide produced from the fusion sequence comprises an N-terminal anchor polypeptide and a C-terminal antibody polypeptide or antibody polypeptide fragment.

56. The method of any of claims 48-55, wherein the Yarrowia cell operating conditions comprise incubation at a low temperature.

57. The method of claim 56, wherein the low temperature comprises a temperature between about 15 degrees Celsius and 25 degrees Celsius.

58. The method of claim 56 or 57, wherein the low temperature comprises a temperature of about 20 degrees Celsius.

59. The method of any of claims 56-58, wherein the low induction temperature comprises a temperature of about 16 degrees Celsius.

60. The method of any one of claims 46-59, wherein the Yarrowia cell operating conditions comprise a short time of incubation.

61. The method of claim 60, wherein the short time is about 24 hours or less.

62. The method of claim 60 or 61, wherein the short time is about 16 hours or less.

63. The method of claim 62, wherein the short time is about 16 hours.

64. The method of any one of claims 46-63, wherein the Yarrowia cell operating conditions comprise a low pH culture medium.

65. The method of claim 64 wherein the low pH is a pH of between about 2 and about 4.

66. The method of claim 64 or 65, wherein the low pH is a pH of about 3.

67. The method of any of claims 46-65, wherein the Yarrowia cell operating conditions comprise high aeration conditions.

68. The method of claim 67, wherein the high aeration conditions comprise incubation in a shake flask.

69. The method of any one of claims 46-68, wherein the Yarrowia cell operating conditions comprise incubation in a minimal medium.

70. The method of claim 69, wherein the minimal medium is a medium that lacks yeast extract, bactopeptone, or both.

71. The method of any one of claims 41-70, wherein the first vector is integrated into the Yarrowia genome.

72. The method of any of claims 43-46 and 48-71, wherein the second vector is integrated into the Yarrowia genome.

73. The method of any one of claims 41-72, wherein the first Yarrowia cell, the second Yarrowia cell, or both Yarrowia cells expresses a chaperone.

74. The method of claim 73, wherein the chaperone is selected from the group consisting of a protein disulfide isomerase, Kar2/Bip (immunoglobulin binding protein), and combinations thereof.

75. The method of any one of claims 41-74, wherein the anchor polypeptide is selected from the group consisting of: an Aga (mating type A agglutinin)1p polypeptide or fragment thereof, and an Aga2p polypeptide or fragment thereof, or a Sag (S. cerevisiae agglutinin)1p polypeptide or fragment thereof.

76. An antibody polypeptide or antibody polypeptide fragment obtained by the method of any one of claims 41-75.

77. A method of selecting a Yarrowia cell comprising an antibody polypeptide, or antibody polypeptide fragment, that binds a target polypeptide, the method comprising:
contacting a parent Yarrowia cell with the test polypeptide, wherein the parent Yarrowia cell displays on its surface a molecule comprising a first antibody polypeptide or a first antibody polypeptide fragment and wherein the parent Yarrowia cell comprises a first expression cassette comprising a first nucleic acid sequence comprising a nucleotide sequence encoding the first antibody polypeptide or the first antibody polypeptide fragment, wherein the first antibody polypeptide fragment comprises an antibody variable (V) region; and
selecting the parent Yarrowia cell if the displayed molecule binds the target polypeptide.

78. The method of claim 77, wherein the parent Yarrowia cell and the second Yarrowia cell further comprise a second expression cassette comprising a second nucleic acid sequence comprising a nucleotide sequence encoding a second antibody polypeptide or a second antibody polypeptide fragment, and wherein the molecule further comprises the second antibody polypeptide or the second antibody polypeptide fragment wherein the second antibody polypeptide fragment comprises an antibody variable (V) region.

79. The method of claim 77 or 78, wherein the parent Yarrowia cell is produced by the method of any of claims 41-75.

80. The method of any of claims 77-79, further comprising:
isolating the first expression cassette from the selected parent Yarrowia, cell;
introducing one or more changes in the nucleotide sequence to generate a modified expression cassette;
introducing the modified expression cassette into a second Yarrowia cell that lacks the first expression cassette to generate a modified Yarrowia cell;
induction incubating the modified Yarrowia cell;
contacting the modified Yarrowia cell with the target polypeptide; and
selecting the modified Yarrowia cell if it binds the target polypeptide with greater affinity or avidity than the parent Yarrowia cell.

81. The method of claim 79, wherein the induction incubation is under one or more Yarrowia operating conditions.

82. A kit comprising the cell of any one of claims 1-40.

83. The kit of claim 82, further comprising written instructions for use of the cell.
